SEQUENCE LISTING (29 sequences)

SEQ ID NO: 1 - DNA (Genomic DNA), 795 bp; single-stranded, linear
  CDS 16-789
  hypothetical: no
  anti-sense: no
aggagggtttttcatatgccaatcgtgcagaacatccaggggcaaatggtacatcaggccatatcacctagaactttaaatgcatgggtaaaagtagtagaagagaaggctttcagcccagaagtgatacccatgttttcagcattatcagaaggagccaccccacaagatttaaacaccatgctaaacacagtggggggacatcaagcagccatgcaaatgttaaaagagaccatcaatgaggaagctgcagaatgggatagagtgcatccagtgcatgcagggcctattgcaccaggccagatgagagaaccaaggggaagtgacatagcaggaactactagtacccttcaggaacaaataggatggatgacaaataatccacctatcccagtaggagaaatttataaaagatggataatcctgggattaaataaaatagtaagaatgtatagccctaccagcattctggacataagacaaggaccaaaggaaccctttagagactatgtagaccggttctataaaactctaagagccgagcaagcttcacaggaggtaaaaaattggatgacagaaaccttgttggtccaaaatgcgaacccagattgtaagactattttaaaagcattgggaccagcggctacactagaagaaatgatgacagcatgtcagggagtaggaggacccaaaaatcaacaattattatccttatgggggtgtaaagggaaacttgtttgttatacttccgttaaatggaatggacccggccataaggcaagagttttgtaataa

SEQ ID NO: 2 - protein, 258 aa
MPIVQNIQGQMVHQAISPRTLNAWVKVVEEKAFSPEVIPMFSALSEGATPQDLNTMLNTVGGHQAAMQMLKETINEEAAEWDRVHPVHAGPIAPGQMREPRGSDIAGTTSTLQEQIGWMTNNPPIPVGEIYKRWIILGLNKIVRMYSPTSILDIRQGPKEPFRDYVDRFYKTLRAEQASQEVKNWMTETLLVQNANPDCKTILKALGPAATLEEMMTACQGVGGPKNQQLLSLWGCKGKLVCYTSVKWNGPGHKARVL

SEQ ID NO: 3 - DNA (Genomic DNA), 795 bp; single-stranded, linear
  CDS 16-789
  hypothetical: no
  anti-sense: no
aggagggtttttcatatgccaatcgtgcagaacatccaggggcaaatggtacatcaggccatatcacctagaactttaaatgcatgggtaaaagtagtagaagagaaggctttcagcccagaagtgatacccatgttttcagcattatcagaaggagccaccccacaagatttaaacaccatgctaaacacagtggggggacatcaagcagccatgcaaatgttaaaagagaccatcaatgaggaagctgcagaatgggatagagtgcatccagtgcatgcagggcctattgcaccaggccagatgagagaaccaaggggaagtgacatagcaggaactactagtacccttcaggaacaaataggatggatgacaaataatccacctatcccagtaggagaaatttataaaagatggataatcctgggattaaataaaatagtaagaatgtatagccctaccagcattctggacataagacaaggaccaaaggaaccctttagagactatgtagaccggttctataaaactctaagagccgagcaagcttcacaggaggtaaaaaattggatgacagaaaccttgttggtccaaaatgcgaacccagattgtaagactattttaaaagcattgggaccagcggctacactagaagaaatgatgacagcatgtcagggagtaggaggacccaaaaatcaacaaagattaaatttatgggggtgtaaagggaaacttatttgttatacttccgttaaatggaatggacccggccataaggcaagagttttgtaataa

SEQ ID NO: 4 - protein, 258 aa
MPIVQNIQGQMVHQAISPRTLNAWVKVVEEKAFSPEVIPMFSALSEGATPQDLNTMLNTVGGHQAAMQMLKETINEEAAEWDRVHPVHAGPIAPGQMREPRGSDIAGTTSTLQEQIGWMTNNPPIPVGEIYKRWIILGLNKIVRMYSPTSILDIRQGPKEPFRDYVDRFYKTLRAEQASQEVKNWMTETLLVQNANPDCKTILKALGPAATLEEMMTACQGVGGPKNQQRLNLWGCKGKLICYTSVKWNGPGHKARVL

SEQ ID NO: 5 - DNA (Genomic DNA), 795 bp; single-stranded, linear
  CDS 16-789
  hypothetical: no
  anti-sense: no
aggagggtttttcatatgccaatcgtgcagaacatccaggggcaaatggtacatcaggccatatcacctagaactttaaatgcatgggtaaaagtagtagaagagaaggctttcagcccagaagtgatacccatgttttcagcattatcagaaggagccaccccacaagatttaaacaccatgctaaacacagtggggggacatcaagcagccatgcaaatgttaaaagagaccatcaatgaggaagctgcagaatgggatagagtgcatccagtgcatgcagggcctattgcaccaggccagatgagagaaccaaggggaagtgacatagcaggaactactagtacccttcaggaacaaataggatggatgacaaataatccacctatcccagtaggagaaatttataaaagatggataatcctgggattaaataaaatagtaagaatgtatagccctaccagcattctggacataagacaaggaccaaaggaaccctttagagactatgtagaccggttctataaaactctaagagccgagcaagcttcacaggaggtaaaaaattggatgacagaaaccttgttggtccaaaatgcgaacccagattgtaagactattttaaaagcattgggaccagcggctacactagaagaaatgatgacagcatgtcagggagtaggaggaccacaaaatcaacaacttttaaatttatgggggtgtagagggaaagctatttgttatacttccgttcaatggaatggacccggccataaggcaagagttttgtaataa

SEQ ID NO: 6 - protein, 258 aa
MPIVQNIQGQMVHQAISPRTLNAWVKVVEEKAFSPEVIPMFSALSEGATPQDLNTMLNTVGGHQAAMQMLKETINEEAAEWDRVHPVHAGPIAPGQMREPRGSDIAGTTSTLQEQIGWMTNNPPIPVGEIYKRWIILGLNKIVRMYSPTSILDIRQGPKEPFRDYVDRFYKTLRAEQASQEVKNWMTETLLVQNANPDCKTILKALGPAATLEEMMTACQGVGGPQNQQLLNLWGCRGKAICYTSVQWNGPGHKARVL

SEQ ID NO: 7 - DNA (Genomic DNA), 378 bp; single-stranded, linear
  CDS 16-375
  hypothetical: no
  anti-sense: no
aggagggtttttcatatgagcacgaatcctaaacctcaaagaaaaaccaaacgtaacaccaaccgtcgcccacaggacgtcaagttcccgggtggcggtcagatcgttggtggagtttacttgttgccgcgcaggggccctagattgggtgtgcgcgcgacgaggaagacttccgagcggtcgcaacctcgaggtagacgtcagcctatccccaaggtgcgtcggccggagggcaggacctgggctcagcccgggtacccttggcccctctatggcaatgagggttgcgggtgggcgggatggctcctgtctccccgtggctctcggcctagctggggccccacagacccccggcgtaggtcgcgcaatttgggttaa

SEQ ID NO: 8 - protein, 120 aa
MSTNPKPQRKTKRNTNRRPQDVKFPGGGQIVGGVYLLPRRGPRLGVRATRKTSERSQPRGRRQPIPKVRRPEGRTWAQPGYPWPLYGNEGCGWAGWLLSPRGSRPSWGPTDPRRRSRNLG

SEQ ID NO: 9 - DNA (Genomic DNA), 378 bp; single-stranded, linear
  CDS 16-375
  hypothetical: no
  anti-sense: no
aggagggtttttcatatgagcacgaatcctaaacctcaaagaaaaaccaaacgtaacaccaaccgtcgcccacaggacgtcaagttcccgggtggcggtcagatcgttggtggagtttacttgttgccgcgcaggggccctagattgggtgtgcgcgcgacgaggaagacttccgagcggtcgcaacctcgaggtagacgtcagcctatccccaaggcacgtcggcccgagggcaggacctgggctcagcccgggtacccttggcccctctatggcaatgagggttgcgggtgggcgggatggctcctgtctccccgtggctctcggcctagctggggccccacagacccccggcgtaggtcgcgcaatttgggttaa

SEQ ID NO: 10 - protein, 120 aa
MSTNPKPQRKTKRNTNRRPQDVKFPGGGQIVGGVYLLPRRGPRLGVRATRKTSERSQPRGRRQPIPKARRPEGRTWAQPGYPWPLYGNEGCGWAGWLLSPRGSRPSWGPTDPRRRSRNLG

SEQ ID NO: 11 - DNA (Genomic DNA), 378 bp; single-stranded, linear
  CDS 16-375
  hypothetical: no
  anti-sense: no
aggagggtttttcatatgagcacgaatcctaaacctcaaagaaaaaccaaacgtaacaccaaccgtcgcccacaggacgtcaagttcccgggtggcggtcagatcgttggtggagtttacttgttgccgcgcaggggccctagattgggtgtgcgcgcgacgaggaagacttccgagcggtcgcaacctcgaggtagacgtcagcctatccccaaggaccgtcggtccacgggcaagtcctggggtaagcccgggtacccttggcccctctatggcaatgagggttgcgggtgggcgggatggctcctgtctccccgtggctctcggcctagctggggccccacagacccccggcgtaggtcgcgcaatttgggttaa

SEQ ID NO: 12 - protein, 120 aa
MSTNPKPQRKTKRNTNRRPQDVKFPGGGQIVGGVYLLPRRGPRLGVRATRKTSERSQPRGRRQPIPKDRRSTGKSWGKPGYPWPLYGNEGCGWAGWLLSPRGSRPSWGPTDPRRRSRNLG

SEQ ID NO: 13 - DNA (Genomic DNA), 378 bp; single-stranded, linear
  CDS 16-375
  hypothetical: no
  anti-sense: no
aggagggtttttcatatgagcacgaatcctaaacctcaaagaaaaaccaaacgtaacaccaaccgtcgcccacaggacgtcaagttcccgggtggcggtcagatcgttggtggagtttacttgttgccgcgcaggggccctagattgggtgtgcgcgcgacgaggaagacttccgagcggtcgcaacctcgaggtagacgtcagcctatccccaaggcacgtcggtccgagggcaggtcctgggctcagcccgggtacccttggcccctctatggcaatgagggttgcgggtgggcgggatggctcctgtctccccgtggctctcggcctagctggggccccacagacccccggcgtaggtcgcgcaatttgggttaa

SEQ ID NO: 14 - protein, 120 aa
MSTNPKPQRKTKRNTNRRPQDVKFPGGGQIVGGVYLLPRRGPRLGVRATRKTSERSQPRGRRQPIPKARRSEGRSWAQPGYPWPLYGNEGCGWAGWLLSPRGSRPSWGPTDPRRRSRNLG

SEQ ID NO: 15 - DNA (Genomic DNA), 381 bp; single-stranded, linear
  CDS 16-375
  hypothetical: no
  anti-sense: no
aggagggtttttcatatgcctattcatcatcatcatcatcatggcccgggctccgtcactgtgtcccatcctaacatcgaggaggttgctctgtccaccaccggagagatccccttttacggcaaggctatccccctcgaggtgatcaaggggggaagacatctcatcttctgccactcaaagaagaagtgcgacgagctcgccgcgaagctggtcgcattgggcatcaatgccgtggcctactaccgcggtcttgacgtgtctgtcatcccgaccagcggcgatgttgtcgtcgtgtcaaccgatgctctcatgactggctttaccggcgacttcgactcggtgatagactgcaatacgggtaccgagctcgaattctaa

SEQ ID NO: 16 - protein, 121 aa
MPIHHHHHHGPGSVTVSHPNIEEVALSTTGEIPFYGKAIPLEVIKGGRHLIFCHSKKKCDELAAKLVALGINAVAYYRGLDVSVIPTSGDVVVVSTDALMTGFTGDFDSVIDCNTGTELEF

SEQ ID NO: 17 - DNA (Genomic DNA), 774 bp; single-stranded, linear
  CDS 16-771
  hypothetical: no
  anti-sense: no
aggagggtttttcatatgtcccctatactaggttattggaaaattaagggccttgtgcaacccactcgacttcttttggaatatcttgaagaaaaatatgaagagcatttgtatgagcgcgatgaaggtgataaatggcgaaacaaaaagtttgaattgggtttggagtttcccaatcttccttattatattgatggtgatgttaaattaacacagtctatggccatcatacgttatatagctgacaagcacaacatgttgggtggttgtccaaaagagcgtgcagagatttcaatgcttgaaggagcggttttggatattagatacggtgtttcgagaattgcatatagtaaagactttgaaactctcaaagttgattttcttagcaagctacctgaaatgctgaaaatgttcgaagatcgtttatgtcataaaacatatttaaatggtgatcatgtaacccatcctgacttcatgttgtatgacgctcttgatgttgttttatacatggacccaatgtgcctggatgcgttcccaaaattagtttgttttaaaaaacgtattgaagctatcccacaaattgataagtacttgaaatccagcaagtatatagcatggcctttgcagggctggcaagccacgtttggtggtggcgaccatcctccaaaatcggatctggttccgcgtggatccgacgtcaagttcccgggtggcggtcagatcgttggtggagtttacttgttgccgcgcagggaattcatcgtgactgactga

SEQ ID NO: 18 - protein, 252 aa
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLNGDHVTHPDFMLYDALDVVLYMDPMCLDAFPKLVCFKKRIEAIPQIDKYLKSSKYIAWPLQGWQATFGGGDHPPKSDLVPRGSDVKFPGGGQIVGGVYLLPRREFIVTD

SEQ ID NO: 19 - DNA (Genomic DNA), 31 bp; single-stranded, linear
  hypothetical: no
  anti-sense: no
ccaaaattaccatatgccaatcgtgcagaac

SEQ ID NO: 20 - DNA (Genomic DNA), 33 bp; single-stranded, linear
  hypothetical: no
  anti-sense: no
gacccggccataaggcaagagttttgtaataag

SEQ ID NO: 21 - DNA (Genomic DNA), 34 bp; single-stranded, linear
  hypothetical: no
  anti-sense: yes
gatccttattacaaaactcttgccttatggccgg

SEQ ID NO: 22 - DNA (Genomic DNA), 28 bp; single-stranded, linear
  hypothetical: no
  anti-sense: no
gctcgcatatgagcacgattcccaaacc

SEQ ID NO: 23 - DNA (Genomic DNA), 32 bp; single-stranded, linear
  hypothetical: no
  anti-sense: yes
gacgaattcttaacccaaattgcgcgacctac

SEQ ID NO: 24 - DNA (Genomic DNA), 66 bp; single-stranded, linear
  hypothetical: no
  anti-sense: no
gatccgacgtcaagttcccgggtggcggtcagatcgttggtggagtttacttgttgccgcgcaggg

SEQ ID NO: 25 - DNA (Genomic DNA), 66 bp; single-stranded, linear
  hypothetical: no
  anti-sense: yes
aattccctgcgcggcaacaagtaaactccaccaacgatctgaccgccacccgggaacttgacgtcg

SEQ ID NO: 26 - DNA (Genomic DNA), 28 bp; single-stranded, linear
  hypothetical: no
  anti-sense: no
ggaattccatatgtcccctatactaggt

SEQ ID NO: 27 - DNA (Genomic DNA), 26 bp; single-stranded, linear
  hypothetical: no
  anti-sense: yes
cggaattctcacctgcgcggcaacaa

SEQ ID NO: 28 - DNA (Genomic DNA), 52 bp; single-stranded, linear
  hypothetical: no
  anti-sense: no
tatgcctattcatcatcatcatcatcatggcccgggaattctaagtaagtag

SEQ ID NO: 29 - DNA (Genomic DNA), 54 bp; single-stranded, linear
  hypothetical: no
  anti-sense: yes
gatcctacttacttagaattcccgggccatgatgatgatgatgatgaataggca

(19) United States
(12) Patent Application Publication
(10) Pub. No.: US 2004/0214163 A1
(43) Pub. Date: Oct. 28, 2004

(54) Methods and systems for producing recombinant viral antigens

(76) Inventors: Suzanne Zebedee, Carlsbad, CA (US); Genevieve Inchauspe, Lyon (FR); Marc S. Nasoff, San Diego, CA (US); Alfred M. Prince, Pound Ridge, NY (US); Torsten B. Helting, San Francisco, CA (US); Michael F. Nunn, Washington, DC (US)

Correspondence Address: Joseph E. Mueth, Esq., Joseph E. Mueth Law Corporation, 8th Floor, 225 South Lake Avenue, Pasadena, CA, 91101, US

(21) Appl. No.: 10/677,956

(22) Filed: Oct. 1, 2003

Related U.S. Application Data
(62) Division of application No. 08/931,855, filed on Sep. 16, 1997, now Pat. No. 6,692,751.
Application No. 08/931,855 is a continuation-in-part of application No. 08/563,733, filed on Nov. 28, 1995, now abandoned.
Application No. 08/563,733 is a division of application No. 08/049,531, filed on Apr. 20, 1993, now Pat. No. 5,470,720.
Application No. 08/049,531 is a division of application No. 07/344,237, filed on Apr. 26, 1989, now Pat. No. 5,204,259.
Application No. 07/344,237 is a continuation-in-part of application No. 07/191,229, filed on May 6, 1988, now abandoned.

(51) Int. Cl. C12Q 1/70; C07H 21/04; C07K 14/16

(52) U.S. Cl. 435/5; 435/69.3; 435/320.1; 435/325; 530/350; 536/23.72

(57) ABSTRACT

The present invention relates to recombinant expression vectors which express segments of deoxyribonucleic acid that encode recombinant HIV and HCV antigens. These recombinant expression vectors are transformed into host cells and used in a method to express large quantities of these antigens. The invention also provides compositions containing certain of the isolated antigens, diagnostic systems containing these antigens and methods of assaying body fluids to detect the presence of antibodies against the antigens of the invention.

This is a continuation-in-part application of co-pending Ser. No. 563,733, filed Nov. 28, 1995, which is a division of Ser. No. 049,531, filed Apr. 20, 1993, U.S. Pat. No. 5,470,720, which is a division of Ser. No. 344,237, filed Apr. 26, 1989, U.S. Pat. No. 5,204,259, which is a continuation-in-part of Ser. No. 191,229, filed May 6, 1988, abandoned, Ser. No. 206,499, filed Jun. 13, 1988, abandoned and Ser. No. 258,016, filed Oct. 14, 1988, abandoned; and of co-pending Ser. No. 272,271, filed Jul. 8, 1994, which is a continuation of Ser. No. 616,369, filed Nov. 21, 1990, abandoned, which is a continuation-in-part of Ser. No. 573,643, filed Aug. 27, 1990, abandoned; the disclosures of which are incorporated herein by reference.

### FIELD OF THE INVENTION

The present invention relates to recombinant expression vectors which have segments of deoxyribonucleic acid (DNA) that encode recombinant HIV and HCV antigens operatively linked to the sequence AGGAGGGTTTTTCAT (nucleotides 1 to 15 of SEQ ID NO: 1) to control expression of the antigens. These recombinant expression vectors are transformed into host cells and used in a method to express large quantities of these antigens. The invention also provides compositions containing certain of the isolated antigens, diagnostic systems containing these antigens and methods of assaying body fluids to detect the presence of antibodies against the antigens of the invention.

### BACKGROUND OF THE INVENTION

The development of immunoassays for the detection of antibodies has been limited by difficulties in producing sufficient quantities of specific antigens that are essentially free of immunoreactive contaminants. The presence of contaminants that react with antibodies present in patient samples results in lower assay specificity and sensitivity and an increase in false positive results. The production of large amounts of antigen enables easier purification of antigen having a higher degree of purity and thus less immunoreactive contaminants.

The present invention overcomes the difficulties by providing a simple and highly efficient expression system that allows for the production of large quantities of antigens. The invention relies on the efficient expression resulting from the inclusion of the nucleotide sequence AGGAGGGTTTTTCAT directly upstream from the ATG codon which marks the start of translation.

The invention is particularly useful for the expression of viral antigens of Human Immunodeficiency Virus (HIV) and Hepatitis C Virus (HCV).

HIV is the causative agent of Acquired Immunodeficiency Syndrome (AIDS). The nucleic acid sequence of the HIV proviral genome has been deduced and the location of various protein coding regions within the viral genome has been determined. Of particular interest to the present invention are the portions of the HIV genome known in the art as the gag and env regions. The gag region encodes a precursor protein that is cleaved and processed into three mature proteins, p17, p24 and p15. The HIV p24 protein has an apparent relative molecular weight of about 24,000 daltons and is known in the art as the HIV core antigen because it forms the viral capsid. Also of interest is the env region which encodes the envelope glycoproteins gp120 and gp41, which are required for viral entry into the cell. The first step in infection is the formation of a complex of gp120, gp41 and the cellular CD4 protein, binding the virus particle to the cell. The formation of this complex appears to alter the confirmation of gp41, allowing its interaction with a second cellular protein “fusin”, an interaction required for HIV entry into the cell.

The p24 antigen of HIV is of particular interest because studies have indicated that the first evidence of anti-HIV antibody formation (sero-conversion) in infected individuals is the appearance of antibodies induced by the p24 antigen, i.e., anti-p24 antibodies. In addition, recent studies have reported that p24 protein can be detected in blood samples even before the detection of anti p24 antibodies. Detecting the presence of either the p24 protein or anti-p24 antibodies therefore appears to be the best approach to detecting HIV infection at the earliest point in time. Furthermore, the p24 antigen reappears in the blood of infected individuals concomitant with the decline of anti-p24 antibody in patients showing the deterioration in their clinical condition that accompanies transition into full-blown AIDS. Thus, the p24 antigen can serve as an effective prognostic marker in patients undergoing therapy.

Most cases of Non-A, non-B hepatitis (NANBH) are caused by the transmissible virus now designated as hepatitis C virus (HCV). Isolates of HCV nucleic acids have been obtained and completely characterized at the sequence level. The HCV genome is comprised of a plus strand RNA molecule that codes for a single polyprotein which is cleaved to produce functionally distinct structural and nonstructural HCV proteins. Structural proteins include the capsid and envelope proteins which form the viral particle. Nonstructural proteins, such as helicase and RNA-directed RNA polymerase are required for viral function.

Some HCV gene products, or portions thereof have been expressed as fusion products. The HCV antigen C-100-3, derived from portions of the nonstructural genes designated NS3 and NS4, has been expressed as a fusion protein and used to detect anti-C-100-3 antibodies in patients with various forms of NANB hepatitis. See, for example, Kuo et al, Science, 244:362-364 (1989) and International Application No. PCT/US88/04125. A diagnostic assay based on C-100-3 antigen is commercially available from Ortho Diagnostics, Inc. (Raritan, N.J.). However, the C-100-3 antigen-based immunoassay has been reported to preferentially detect antibodies in sera from chronically infected patients. C-100-3 seroconversion generally occurs from four to six months after the onset of hepatitis, and in some cases C-100-3 fails to detect any antibody where an NANBV infection is present. Alter et al, New Eng. J. Med., 321:1538-39 (1989); Alter et al, New Eng. J. Med., 321:1494-1500 (1989); and Weineret al, Lancet, 335:1-3 (1990). McFarlane et al, Lancet. 335:754-757 (1990), described false positive results when the C-100-3-based immunoassay was used to measure antibodies in patients with autoimmune chronic active hepatitis. In addition, Grey et al., Lancet, 335:609-610 (1990), describe false positive results using C-100-3-based immunoassay on sera from patients with liver disease caused-by a variety of conditions other than HCV. Houghton et al., U.S. Pat. No. 5,350,671, have disclosed a series of fusion proteins which include amino acids from parts of various structural and nonstructural HCV gene products fused to superoxide dismutase (SOD), many of which have no immunogenic activity when tested against HCV positive antisera.

The present invention provides compositions of recombinantly produced HIV and HCV antigens, free of bacterial and other viral components, thus enabling the detection of HIV and HCV antibodies with improved accuracy and sensitivity. The present invention also enables high yield expression of these antigens alone or as fusion proteins.

### SUMMARY OF THE INVENTION

The present invention is directed to recombinant expression vectors which comprise a first nucleic acid having the sequence AGGAGGGTTTTTCAT operatively linked to a second nucleic acid having a sequence encoding an HIV or HCV antigen.

The preferred vectors of the inventions are pGEX7 derivatives. The pGEX7 vector contains the first nucelic acid sequence (AGGAGGGTTTTTCAT). Thus, the second nucleic acid encoding the HIV antigen or HCV antigen is operatively linked to pGEX7-derived first nucelic acid.

In addition to the recombinant expression vectors, the present invention includes host cells comprising these vectors, the recombinant HIV and HCV antigens produced by treating the host cells of the invention for a time and under conditions to cause expression of the antigen, the HIV and HCV antigens produced by this method and compositions comprising a recombinantly-produced HIV or HCV antigen of the invention. The compositions can be essentially free of procaryotic antigens or other viral-related proteins of the respective antigens.

The HIV antigen of the invention comprises three domans which are optionally joined by 1 to 5 linker amino acids. The first domain has a nucleotide sequence which encodes amino acids 1-225 of an HIV p24 antigen, the second domain has a nucleotide sequence which encodes an HIV gp41 antigen (or antigenic fragment thereof), and the third domain has a nucleotide sequence which encodes amino acids 224-232 of an HIV p24 antigen. In preferred embodiments the HIV antigen is encoded by amino acids 1-258 of SEQ ID NO: 2, 4 or 6. These preferred HIV antigens are expressed from the vectors pGEXp24gp41-ANT, pGEXp24gp41-MVP and pGEXp24gp41-X84328, respectively.

The HCV antigens of the invention are the HCV capsid antigen, the HCV non-structural 794 antigen and the HCV CAP-B antigen. In preferred embodiments, the HCV capsid antigen is encoded by amino acids 1-120 from an HCV strain, and more preferably are encoded by amino acids 1-120 of SEQ ID NO:8, 10, 12 or 14. The preferred HCV capsid antigens are expressed from the vectors pGEX-C120H-V68, pGEX-C120H, pGEX-C120H-IS02 and pGEX-C120H-IS03, respectively. In preferred embodiments the HCV non-structural 794 antigen is encoded by the amino acids of SEQ ID NO: 16 or the corresponding sequence from another HCV strain. The antigen of SEQ ID NO: 16 is preferably expressed from pGEX-NS3-794. The CAP-B antigen is encoded by the amino acids of SEQ ID NO: 18 or the corresponding sequence from another HCV strain. The antigen of SEQ ID NO: 18 is preferably expressed from pGEX-CAP-B.

Another aspect of the invention is directed to a diagnostic kit comprising an amount of a HIV antigen or HCV antigen composition of the invention sufficient to perform at least one assay.

Yet another aspect of the invention provides a method of assaying a body fluid sample for the presence of antibodies against an HIV or HCV antigen which comprises:

a) forming an immunoreaction admixture by admixing the body fluid sample with a composition of the invention;

b) maintaining the immunoreaction admixture for a time period sufficient for antibodies present against the desired antigen to immunoreact with the antigen and to form an immunoreaction product; and

c) detecting the presence of any immunoreaction product formed and thereby the presence of the desired antibodies.

The method of claim 15, wherein said detecting in step (c) can further comprise the steps of:

(i) admixing the immunoreaction product with a labeled specific binding agent to form a labeling admixture, wherein the labeled specific binding agent comprises a specific binding agent and a label;

(ii) maintaining the labeling admixture for a time period sufficient for any immunoreaction product present to bind with the labeled specific binding agent to form a labeled product; and

(iii) detecting the presence of any labeled product formed, and thereby the presence of the immunoreaction product.

In preferred embodiments, the specific binding agent can be Protein A, anti-human IgG or anti-human IgM and the label can be biotin, an enzyme, a lanthanide chelate or a radioactive isotope.

Further still, another embodiment of the invention is directed to a composition comprising the HCV capsid antigen of the invention and the HCV nonstructural 794 antigen of the invention which is essentially free of procaryotic antigens and other HCV-related proteins. These compositions can be provided as diagnostic kits and used in the methods of assaying a body fluid to detect antibodies against an HCV capsid antigen or an HCV nonstructural antigen as described above.

### BRIEF SUMMARY OF THE DRAWINGS

FIG. 1 illustrates the plasmid pGEXp24 for expressing recombinant HIV p24 protein in E. coli. The recombinant DNAs manipulated and produced by the construction process are indicated in the figure by the circles. The construction proceeds by a series of steps as indicated by the arrows connecting the circles in the figure and as described in detail in Example 1. Landmark and utilized restriction enzyme recognition sites are indicated on the circles by labeled lines intersecting the circles. The relative location of individual genes and their direction of transcription are indicated by the labeled arrows inside the circles.

FIG. 2 illustrates the HIV p24-gp41 hybrid proteins obtained after purification from induced bacterial cultures previously transformed with pGEXp24gp41 of U.S. Pat. No. 5,470,720 or with pGEXp24gp41-ANT, pGEXp24gp41-MVP or pGEXp24gp41-X84328 of the present invention.

FIG. 3 illustrates the HCV 1-120 capsid antigen (strain Hutch) with an amino acid substitution of valine for alanine at residue 68 after purification from induced bacterial cultures previously transformed with pGEX-C120H-V68 of the present invention.

FIG. 4 illustrates the HCV NS3-794 antigen (strain Hutch) after purification from induced bacterial cultures previously transformed with pGEX7-NS3-794 of the present invention.

FIG. 5 illustrates ELISAs of serially diluted HIV positive antiserum using polystyrene plates coated with (A) p24-gp41 recombinant protein of U.S. Pat. No. 5,470,720; (B) p24-gp41 Subtype O ANT recombinant protein; (C) p24-gp41 Subtype O MVP5180 recombinant protein; and (D) p24-gp41 Subtype O X84328 recombinant protein.

FIG. 6 illustrates the immune reactivity in an ELISA of a combination of the recombinant proteins of FIGS. 3 and 4 with the well-characterized, commercially available Boston Biomedica PHV901 seroconverter serum from an individual who developed HCV infection.

FIG. 7 illustrates the immune reactivity in an ELISA of a combination of the recombinant proteins of FIGS. 3 and 4 with the well-characterized, commercially available Boston Biomedica PHV902 seroconverter serum from an individual who developed HCV infection.

FIG. 8 illustrates the immune reactivity in an ELISA of a combination of the recombinant proteins of FIGS. 3 and 4 with the well-characterized, commercially available Boston Biomedica PHV903 seroconverter serum from an individual who developed HCV infection.

### DETAILED DESCRIPTION OF THE INVENTION

A. Definitions

Amino acid: All amino acid residues identified herein are in the natural L-configuration. All abbreviations for amino acid residues are in keeping with the standard polypeptide nomenclature, J. Biol. Chem. 243: 3557-3559 (1969). It should be noted that all amino acid residue sequences, typically referred to herein as “residue sequences” are represented herein by formulae whose left to right orientation is in the conventional direction of amino terminus to carboxy-terminus.

Nucleotide: a monomeric unit of DNA or RNA consisting of a sugar moiety (pentose) a phosphate and a nitrogenous heterocyclic base. The base is linked to the sugar moiety via the glycoside carbon (1′ carbon of the pentose) and that combination of base and sugar is a nucleoside. When the nucleoside contains a phosphate group bonded to the 3′ or 5′ position of the pentose, it is referred to as a nucleotide. A sequence of operatively linked nucleotides is typically referred to herein as a “base sequence” and it is represented herein by the formula whose left to right orientation is in the conventional direction of 5′ terminus to 3′ terminus.

Base pair (bp): a partnership of adenine (A) with thymine (T), or of cytosine (C) with guanine (G) in a double stranded DNA molecule.

Antigen: a protein or polypeptide portion thereof which is immunologically identifiable. By immunologically identifiable is meant that the protein or polypeptide reacts specifically with naturally occurring or synthetically derived antibodies to form a complex of bound antibody and antigen.

Operatively linked: the juxtaposition of sequence elements, regulatory elements, control sequences and the like with coding sequences for a gene product, wherein the elements so described are joined to one another in a relationship permitting them to function in their intended manner, e.g. to control expression. A control sequence operatively linked to a coding sequence is spatially joined in such a way that expression of the coding sequence is achieved under conditions compatible with the control sequences. A second coding sequence may be operatively linked to an expressed first coding sequence such that the regulatory elements and control sequences of the first coding region govern expression of the second coding sequence as well. In the present invention, operatively linked coding sequences are juxtaposed such that a single expression product is produced which comprises regions from each of the coding sequences.

HIV antigen: As referred to in the current invention, HIV antigen means an HIV p24gp41 hybrid protein which comprises an amino acid sequence from gp41 flanked on its amino terminus by amino acids 1-225 of a HIV p24 protein and on its carboxy terminus by amino acids 224-232 of a HIV p24 protein. In some instances, the sequences of each protein domain can be joined by 1-5 linker amino acids. Exemplary antigens are expressed by plasmids pGEXp24gp41-ANT, pGEXp24gp41-MVP or pGEXp24gp41-X84328 of the present invention.

HCV antigen: As referred to herein, HCV antigen means an HCV CAP-B antigen, an HCV 1-120 capsid antigen or an HCV nonstructural 794 antigen. A nonstructural antigen, in the context of HCV means an antigen not derived from capsid or envelope proteins. An HCV CAP-B antigen consists of amino acid residues 1-220 of glutathione-S-transferase, an intermediate polypeptide portion corresponding to residues 221-226 and defining a cleavage site for the protease Thrombin, a polypeptide portion corresponding to residues 227-246 and defining residues 21-40 of an HCV capsid antigen (exemplified by GenBank accession no. M67463) and with or without a carboxy-terminal tail corresponding to residues 247-252. An HCV 1-120 capsid antigen consists of amino acid residues 1 to 120 of an HCV polyprotein. Herein exemplified are an HCV 1-120 capsid antigen derived from HCV strain Hutch and three homologues with various amino acid substitutions. An HCV nonstructural 794 antigen consists of amino acid residues 1-10 having six histidine residues at positions 4 to 9, a nonstructural NS3 antigen of HCV strain Hutch from residue 11 to residue 115 and a six residue tail. The nonstructural NS3 antigen disclose herein correponds to amino acid residues 1352 to 1456 of the amino acid sequence disclosed in GenBank accession no. 130461. Examples of HCV antigens are encoded by plasmids pGEX-C120H-V68, pGEX-C120H, pGEX-C120H-IS02, pGEX-C120H-IS03, pGEX-NS3-794 and pGEX-CAP-B1 of the current invention.

B. Recombinant DNA Molecules

In living organisms, the amino acid residue sequence of a protein or polypeptide is directly related via the genetic code to the DNA sequence of the structural gene that codes for the protein. Thus, a structural gene can be defined in terms of the amino acid residue sequence, i.e., protein or polypeptide for which it codes.

An important and well known feature of the genetic code is its redundancy. That is, for most of the amino acids used to make proteins, more than one coding nucleotide triplet (codon) can code for or designate a particular amino acid residue. Therefore, a number of different nucleotide sequences may code for a particular amino acid residue sequence. Occasionally, a methylated variant of a purine or pyrimidine may be incorporated into a given nucleotide sequence. However, such methylations do not affect the coding relationship in any way.

DNA sequences have other functions as well. Expression of a gene product, i.e. transcription of DNA sequences into ribonucleic acid (RNA) sequences and translation of messenger RNA (mRNA) into sequences of amino acids, depends on DNA nucleotide sequences in addition to those which actually encode the amino acid sequence of interest.

A DNA segment of the present invention comprises a first nucleotide base sequence that defines a ribosome binding site and has a sequence by the formula:

AGGAGGGTTTTTCAT.

The first sequence is joined at its 3′ terminus to the 5′ terminus of a second nucleotide base sequence that defines the structural gene product of interest. Structural gene products may include natural proteins, polypeptides, fusion proteins and proteins to which additional sequences of amino acids with specific functions have been added. Preferred DNA segments are illustrated in SEQ ID NO: 1, 3, 5, 7, 9, 11, 13, 15 and 17 and further include the base sequence TAA or similar sequences representing one or several stop signals, operatively linked to the 3′ terminus of the structural gene. The base sequences are shown conventionally from left to right and in the direction of 5′ terminus to 3′ terminus of the coding sequence using the single letter nucleotide base code (A=Adenine, T=Thymine, C=Cytosine and G=Guanine). Nucleotide bases 1-4 represent the Shine Delgarno sequence (Shine et al. Proc. Natl. Acad. Sci. USA Natl. Acad. Sci. USA Nati Acad. Sci USA 71:1342 (1974)). Bases 1-15 of the above listed sequences define the 15 bases AGGAGGGTTTTTCAT immediately preceding the nucleotide sequence encoding the antigen of interest, said 15 bases positioned immediately upstream of the polylinker cloning site of the ATCC deposited vector pGEX7 referred to herein. The amino acid sequences of the products expressed from the preferred DNA segments are given by SEQ ID NO: 2, 4, 6, 8, 10, 12, 14, 16 and 18.

In one embodiment of this invention, a DNA segment has the nucleotide sequence AGGAGGGTTTTTCAT joined to a nucleotide base sequence that defines an HIV antigen such as an HIV p24-gp41 hybrid protein. The phrase “HIV p24-gp41 hybrid protein” refers to a protein having an amino-terminal HIV p24 polypeptide portion joined by a peptide bond at its carboxy-terminus to an HIV gp41 polypeptide portion followed by another HIV p24 polypeptide portion. In the expressed protein, the first HIV p24 polypeptide portion has an amino acid residue sequence corresponding to residue 2 to residue 225 from one of the sequences shown in SEQ ID NO:2, 4 or 6. The second HIV p24 polypeptide portion has an amino acid sequence corresponding to residues 224 to 232 of an HIV p24 protein, which correspond to residues 250 to 258 of SEQ ID NOS: 2, 4 and 6 for the expressed HIV p24-gp41 hybrid protein.

The HIV gp41 polypeptide portion has an amino acid residue sequence corresponding to a polypeptide capable of immunoreacting with anti-HIV gp41 antibodies, i.e., a polypeptide displaying HIV gp41 antigenicity (an HIV gp41-antigenic polypeptide). Polypeptides displaying HIV gp41 antigenicity are well known in the art. See, for example, the U.S. Pat. No. 4,629,783 to Cosand, U.S. Pat. No. 4,735,896 to Wang et al., and Kennedy et al., Science, 231:1556-1559 (1986).

In preferred embodiments, the HIV gp41 polypeptide portion of the HIV p24-gp41 fusion protein of this invention contains at least 10 amino acid residues, but no more than about 35 amino acid residues, and preferably has a length of about 15 to about 30 residues. A preferred HIV gp41 polypeptide portion of a HIV p24-gp41 hybrid protein has an amino acid residue sequence represented by residue 227 to residue 249 shown in SEQ ID NO:2, by residue 227 to residue 249 shown in SEQ ID NO:4 or by residue 227 to residue 249 shown in SEQ ID NO:6.

In preferred embodiments, that portion of a HIV p24-gp41 hybrid protein encoding DNA segment of this invention that codes for the first HIV p24 polypeptide portion has a nucleotide base sequence corresponding to a sequence that codes for an amino acid residue sequence as shown in SEQ ID NOS:2, 4 and 6 from residue 1 to about residue 225, and more preferably has a nucleotide base sequence corresponding to a base sequence as shown in SEQ ID NOS: 1, 3 and 5 from base 16 to base 690.

In preferred embodiments, that portion of a HIV p24-gp41 hybrid protein encoding DNA segment of this invention that codes for the HIV gp41 polypeptide portion has a nucleotide base sequence corresponding to a sequence that codes for an amino acid residue sequence as shown in SEQ ID NO:2 from residue 227 to residue 249, in SEQ ID NO:4 from residue 227 to residue 249, or in SEQ ID NO:6 from residue 227 to residue 249. More preferably that portion of the DNA segment coding for the HIV gp41 polypeptide portion has a nucleotide base segment corresponding in base sequence to the sequence shown in SEQ ID NO: 1 from base 694 to base 762, in SEQ ID NO:3 from base 694 to base 762, or in SEQ ID NO:5 from base 694 to base 762.

In preferred embodiments, that portion of a HIV p24-gp41 hybrid protein encoding DNA segment of this invention that codes for the second HIV p24 polypeptide portion has a nucelotide base sequence corresponding to a sequence that codes for an amino acid sequence as shown in SEQ ID NOS: 2, 4 and 6 from residue 250 to 258, and more preferably has a nucleotide base sequence corresponding to a base sequence as shown in SEQ ID NOS 1, 3 and 5 from base 763 to base 789.

Several HIV Type I, subtype O conserved sequences are well known. (see, e.g., Cohen et al. Lancet, 345 p. 856, 1995, or GenBank Accession #X84328). In a particularly preferred embodiment, recombinant HIV p24-gp41 hybrid protein is identified by SEQ ID NO:2 and contains an amino terminal p24 polypeptide portion (residues 2-225) followed by a Lys residue as linker amino acid to an intermediate, type O (strain ANT) specific HIV envelope portion (residues 227-249), and a carboxy terminal HIV p24 polypeptide portion (residues 250-258).

A second particularly preferred recombinant HIV p24-gp41 hybrid protein is identified by SEQ ID NO:4, wherein residues 227-249 correspond to a type O specific HIV envelope portion of strain MVP. A third particularly preferred recombinant HIV p24-gp41 hybrid protein is identified by SEQ ID NO:6. In this hybrid protein, the intermediate linker amino acid residue at position 226 is Gln and residues 227-249 correspond to a type O specific HIV envelope portion of strain GenBank X84328.

Most preferably, a HIV p24-gp41 hybrid protein encoding DNA segment of this invention has a nucleotide base sequence corresponding to the sequence shown in SEQ ID NO: 1 from base 1 to base 795, in SEQ ID NO:3 from base 1 to base 795, or in SEQ ID NO:5 from base 1 to base 795.

In another embodiment of this invention, the nucleotide sequence AGGAGGGTTTTTCAT is joined to a nucleotide base sequence that defines the HCV antigen which is an HCV CAP-B fusion protein. The phrase “CAP-B” refers to a recombinant protein having a first glutathione-S-transferase (GST) polypeptide portion joined by a peptide bond at its carboxy terminus to a second intermediate polypeptide portion defining a cleavage site for Thrombin, said second portion joined by a peptide bond at its carboxy terminus to a third polypeptide portion defining an HCV capsid antigen consisting of amino acids 21-40 of an HCV capsid protein and a six residue tail.

The GST portion of a recombinant CAP-B antigen has an amino acid residue sequence corresponding to a sequence as shown in SEQ ID NO: 18 from residue 2 to about residue 220, the amino terminal methionine being cleaved after translation. An intermediate polypeptide portion defining a thrombin cleavage site has the amino acid sequence shown in SEQ ID NO: 18 from residue 221 to residue 226.

SEQ ID NO: 18 illustrates the amino acid sequence of a particularly preferred recombinant CAP-B fusion protein wherein amino acids 1-220 are from GST, residues 221-226 are a cleavage site for protease Thrombin, residues 227 to 246 are from the HCV capsid antigen with the amino acid sequence of residues 21-40 from GenBank accession no. M67463 (strain Hutch) and residues 247 to 252 are a carboxy terminal tail.

In preferred embodiments, that portion of a CAP-B protein encoding DNA segment of this invention that codes for the GST portion has a nucleotide base sequence corresponding to a sequence that codes for an amino acid residue sequence as shown in SEQ ID NO: 18 from about residue 1 to about residue 220 and more preferably has a nucleotide base sequence corresponding to a base sequence as shown in SEQ ID NO: 17 from base 16 to base 675.

In preferred embodiments, that portion of a CAP-B protein encoding DNA segment of this invention that codes for the intermediate polypeptide portion defining a thrombin cleavage site has a nucleotide base sequence corresponding to a sequence that codes for an amino acid residue sequence as shown in SEQ ID NO: 18 from residue 221 to residue 226 and more preferably has a nucleotide base sequence corresponding to a base sequence as shown in SEQ ID NO: 17 from base 676 to base 693.

In preferred embodiments, that portion of a CAP-B protein encoding DNA segment of this invention that codes for the HCV 21-40 capsid portion has a nucleotide base sequence corresponding to a sequence that codes for an amino acid residue sequence as shown in SEQ ID NO: 18 from residue 227 to residue 246 and more preferably has a nucleotide base sequence corresponding to a base sequence shown in SEQ ID NO: 17 from base 694 to base 753.

In a particularly preferred embodiment, the CAP-B protein encoding DNA segment codes for an amino acid residue sequence as shown in SEQ ID NO: 18 from residue 1 to residue 252. Most preferably, a CAP-B protein encoding DNA segment of this invention has a nucleotide base sequence corresponding to the sequence disclosed by SEQ ID NO: 17 from base 1 to base 774, and consists of a ribosome binding site, coding sequence and a stop codon for expression of the HCV strain Hutch CAP-B antigen.

This invention is further embodied by a DNA segment with the nucleotide sequence AGGAGGGTTTTTCAT joined to a nucleotide base sequence that defines the HCV antigen which is an HCV 1-120 capsid antigen. The phrase “capsid antigen” refers to a recombinant protein consisting of amino acids 1-120 of HCV. Preferably, the capsid protein is immunologically related to the Hutch strain of HCV (amino acid sequence 1-120 of GenBank accession no. M67463).

A preferred recombinant HCV capsid antigen is illustrated by SEQ ID NO:8 which represents the structural polypeptide of HCV strain Hutch (amino acid residues 1-120) exhibiting a substitution from Alanine to Valine at amino acid residue 68. Another preferred recombinant HCV capsid antigen is illustrated by SEQ ID NO: 10 which represents the structural polypeptide of HCV strain Hutch. A third recombinant HCV capsid antigen is illustrated by SEQ ID NO: 12 which represents the structural polypeptide of HCV having the amino acid sequence of strain Hutch except wherein amino acid residues 68 to 81 have been substituted by amino acid residues 68 to 81 of the capsid antigen of an HCV genotype 2 isolate. A fourth recombinant HCV capsid antigen is illustrated by SEQ ID NO: 14 which represents the structural polypeptide of HCV having the amino acid sequence of strain Hutch except wherein amino acid residues 68 to 81 have been substituted by amino acid residues 68 to 81 of the capsid antigen of an HCV genotype 3 isolate.

Most preferably, DNA segments of this invention which express preferred HCV 1-120 capsid antigens as illustrated in SEQ ID NOS: 8, 10, 12,. and 14 have nucleotide sequences represented by SEQ ID NOS:7, 9, 11, and 13 (nucleotides 1 to 378) respectively. Represented in each DNA sequence are the ribosome binding site, coding sequence and stop codon. Nucleotides 212 and 259 are the start of 6 nucleotide recognition sites for the StyI restriction endonuclease.

In a final exemplary embodiment, a DNA segment comprises a nucleotide base sequence that defines an HCV antigen which is a recombinant HCV nonstructural 794 antigen. As exemplified herein, “794 antigen” refers to a recombinant protein with the amino acid sequence set forth in SEQ ID NO: 16, which consists of a first 10 amino acid polypeptide region containing a hexahistidine tag (six histidine residues) from amino acid residue 4 to 9, joined by a peptide bond at its carboxy terminus to an NS3 nonstructural antigen (residues 11-115) and a 6 amino acid tail (residues 116 to 121). By NS3 is meant the mature helicase protein of HCV which in strain Hutch corresponds to amino acid residues 1007 to 1615 of the HCV polyprotein. A preferred HCV NS3 nonstructural antigen has the amino acid residue sequence shown in SEQ ID NO: 16 from residue 11 to residue 115, which is that of the Hutch strain of HCV (amino acid sequence 1352-1456 of GenBank accession no. M67463).

The hexahistidine sequence present within the first 10 amino acid sequences exemplifies a “Tag” polypeptide designed to facilitate the purification of the composite synthesis product. Following induction and breakage of cells containing vector encoding a protein with a hexahistidine “Tag”, the protein of interest can be isolated by metal chelate affinity chromatography in accordance with well established procedures (see, eg. Porath et al. Nature, 258 p. 598 (1975)).

In a preferred embodiment, that portion of a recombinant HCV nonstructural 794 antigen encoding DNA segment of this invention that codes for the HCV nonstructural portion has a nucleotide base sequence corresponding to a sequence that codes for an amino acid residue sequence as shown in SEQ ID NO: 16 from residue 11 to residue 115 and more preferably has a nucleotide base sequence corresponding to a base sequence shown in SEQ ID NO: 15 from base 46 to base 360.

In a more preferred embodiment, a recombinant HCV nonstructural 794 antigen encoding DNA segment codes for an amino acid residue sequence as shown in SEQ ID NO: 16 from residue 1 to residue 121. Most preferably, a recombinant HCV nonstructural 794 antigen encoding DNA segment of this invention has a nucleotide base sequence corresponding to the sequence shown in SEQ ID NO: 16 from base 1 to base 381.

In preferred embodiments, a DNA segment of the present invention includes its complimentary DNA segment and is preferably bound thereto, thereby forming a double stranded DNA segment. In addition, it should be noted that a double stranded DNA segment of this invention can have a single stranded cohesive tail at one or both of its termini.

A DNA segment of the present invention can easily be prepared from isolated viruses or other sources by the polymerase chain reaction (PCR) or synthesized by chemical techniques, for example, the phosphotriester method of Matteucci et al. J. Am. Chem. Soc., 103:3185 (1981). (the disclosures of the art cited herein are incorporated herein by reference). Of course, by chemically synthesizing the DNA, any desired modification can be made simply by substituting the appropriate bases for those encoding the native amino acid sequence.

The present invention further contemplates a recombinant DNA (rDNA) that includes a DNA segment of the present invention operatively linked to a vector. A preferred rDNA of the present invention is characterized as being capable of directly expressing, in a compatible host, the gene product of interest. By “directly expressing” it is meant that the mature polypeptide chain of the protein is formed by translation alone as opposed to proteolytic cleavage of two or more terminal amino acid residues from a larger translated precursor protein. Preferred rDNAs of the present invention are derivatives of the pGEX7 expression vector containing the DNA segments of the invention.

As used herein, the term “vector” refers to a DNA molecule capable of autonomous replication in a cell and to which another DNA segment can be operatively linked so as to bring about replication or expression of the attached segment. Typical vectors are plasmids, bacteriophage and the like. Vectors capable of directing the expression of a DNA segment of the invention are referred to herein as “expression vectors”. Thus, a recombinant DNA molecule (rDNA) is a hybrid DNA molecule comprising at least two nucleotide sequences not normally found together in nature. A vector contemplated by the present invention is also least capable of directing replication, and includes a procaryotic replicon (ori), i.e., a DNA sequence having the ability to direct autonomous replication and maintenance of the recombinant DNA molecule extrachromosomally in a procaryotic host cell, such as a bacterial host cell, transformed therewith. Such replicons are well known in the art. In addition, those embodiments that include a procaryotic replicon also typically include a gene whose expression confers drug resistance to a bacterial host transformed therewith. Typical bacterial drug resistance genes for use in these vectors are those that confer resistance to ampicillin or tetracycline. Preferred vectors of the present invention also include a procaryotic promoter capable of directing the expression (transcription and translation) of the gene encoding the HIV or HCV antigen or fusion protein in a bacterial host cell, such as E. coli, transformed therewith. A promoter is an expression control element formed by a DNA sequence that permits binding of RNA polymerase and transcription to occur. Promoter sequences compatible with bacterial hosts are typically provided in plasmid vectors containing convenient restriction sites for insertion of a DNA segment of the present invention. A typical vector is pPL-lambda available from Pharmacia (Piscataway, N.J.).

Although the expression vector pGEX7 has been used as exemplary in producing the proteins described herein, other functionally equivalent expression vectors can be used. Functionally equivalent vectors have the sequence AGGAGGGTTTTTCAT to which coding sequences of interest may be joined, and contain an expression promoter that is inducible by any number of methods such as by temperature shift or by addition of IPTG.

A variety of methods have been developed to operatively link DNA segments to vectors via compatible termini. General recombinant DNA technologies are comprehensively described in a plethora of publications, and for experimental protocols, attention is drawn to the treatise by Maniatis et al. (Molecular Cloning: A Laboratory Manual 2nd edition, Cold Spring Harbor Press (1989)), which is incorporated herein by reference.

Synthetic linkers containing one or more restriction sites provide an alternative method of joining the DNA segments to vectors. The DNA segment, generated by endonuclease digestion or, by some alternate procedure such as primer-directed synthesis via techniques such by PCR (see, eg., supra or, more specialized monographs such as M. J. McPherson, P. Quirke and G. R. Taylor (Eds), “PCR. A Practical Approach”, IRL Press at Oxford University press, Oxford, UK, (1991)) is treated with bacteriophage T4 DNA polymerase or E. coli DNA polymerase I, enzymes that remove protruding 3′ single stranded termini with the 3′-5′ exonucleolytic activities and fill in recessed 3′ ends with their polymerizing activities. The combination of these activities therefore generate blunt-ended DNA segments. The blunted segments are then incubated with a large molar excess of linker molecules in the presence of an enzyme that is able to catalyze the ligation of blunt-ended DNA segments, such as the bacteriophage T4 DNA ligase. Thus, the products of the reaction are DNA segments carrying polymeric linker sequences at their ends. These DNA segments are then cleaved with the appropriate restriction enzyme and ligated to an expression vector that has been cleaved with an enzyme that produces termini compatible with those of the DNA segment. Synthetic linkers containing a variety of restriction endonuclease sites, as well as the restriction endonucleases themselves are commercially available from a number of sources including New England Biolabs (Boston, Mass.).

Also contemplated by the present invention are RNA equivalents of the above described recombinant DNA molecules.

C. Transformed Cells and Cultures

The present invention also relates to a procaryotic host cell transformed with a recombinant DNA molecule of the present invention, preferably an rDNA capable of expressing a recombinant HIV p24-gp41 fusion protein, a recombinant HCV 1-120 capsid protein, a recombinant HCV CAP-B protein or a recombinant HCV nonstructural antigen 794. Bacterial cells are preferred procaryotic host cells and typically are a strain of E. coli, such as, for example, the E. coli strain W3110 or the strain DH5 available from Bethesda Research Laboratories, Inc., Bethesda, Md. Transformation of appropriate cell hosts with a recombinant DNA molecule of the present invention is accomplished by well known methods that typically depend on the type of vector used. With regard to transformation of procaryotic host cells, see, for example, Cohen et al., Proc. Natl. Acad. Sci. USA, 69:2110 (1972); and Maniatis et al., Molecular Cloning, A Laboratory Manual, Cold Spring Harbor Laboratory, Cold Spring Harbor, N.Y. (1982). Successfully transformed cells, i.e., cells that contain a recombinant DNA molecule of the present invention, can be identified by well known techniques. For example, cells resulting from the introduction of an rDNA of the present invention can be cloned to produce monoclonal colonies. Cells from those colonies can be harvested, lysed and their DNA content examined for the presence of the rDNA using a method such as that described by Southern, J. Mol. Biol., 98:503 (1975) or Berent et al., Biotech., 3:208 (1985). In addition to directly assaying for the presence of rDNA, successful transformation can be confirmed by well known immunological methods when the rDNA is capable of directing the expression of a protein from the inserted gene of interest. Samples of cells suspected of being transformed are harvested and assayed for the presence of the encoded HIV or HCV antigen using antibodies specific for the particular antigen of interest. Such antibodies are well known in the art. Thus, in addition to the transformed host cells themselves, the present invention also contemplates a culture of those cells. Nutrient media useful for culturing transformed host cells are well known in the art and can be obtained from several commercial sources.

D. Methods for Producing Recombinant Proteins and Compositions Containing Same

Another aspect of the present invention pertains to a method for producing the HIV and HCV antigens of this invention, more preferably an HIV p24-gp41 fusion protein, an HCV CAP-B protein, an HCV 1-120 capsid protein or an HCV nonstructural antigen 794. The present method entails initiating a culture comprising a nutrient medium containing host cells transformed with a recombinant DNA molecule of the present invention. The culture is maintained for a time period sufficient for the transformed cells to express the HWV or HCV antigen. The expressed protein is then recovered from the culture. However, as is well known in the art, the expressed protein recovered may or may not contain the amino-terminal methionine residue present on the initial translation product depending on cellular processing mechanisms. Methods for recovering an expressed protein from a culture are well known in the art and include fractionation of the protein-containing portion of the culture using well known biochemical techniques. For instance, the methods of gel filtration, gel chromatography, ultrafiltration, electrophoresis, ion exchange, affinity chromatography and the like, such as are known for protein fractionation, can be used to isolate the expressed proteins found in the culture. In addition, immunochemical methods, such as immunoaffinity, immunoadsorption and the like can be performed using well known methods.

E. Recombinant Protein Compositions

In another embodiment, the present invention contemplates a composition containing an HIV or HCV antigen of the invention, including e.g., an HIV p24-gp41 fusion protein, an HCV CAP-B protein, an HCV 1-120 capsid protein or an HCV nonstructural 794 antigen encoded by the DNA segments of the invention or combinations thereof that is essentially free of both procaryotic antigens (i.e. host cell-specific antigens) and other HIV- or HCV-related proteins. By “essentially free” is meant that the ratio of desired HIV or HCV proteins, alone or in combination, to either procaryotic antigen or other HIV- or HCV-related proteins is at least 100:1, and preferably is 1,000:1.

The presence and amount of contaminating protein in a recombinant protein preparation can be determined by well known methods. For example, a sample of the composition is subjected to sodium dodecyl sulfate-polyacrylamide gel electrophoresis (SDS-PAGE) to separate the recombinant protein from any protein contaminants present. The ratio of the amounts of the proteins present in the sample is then determined by densitometric soft laser scanning, as is well known in the art. See Guilian et al., Anal. Biochem., 129:277-287 (1983).

In another embodiment of the invention, the HIV or HCV antigen of the invention is in non-reduced form, e.g., substantially free of sulfhydryl groups because of Cys-Cys bonding that can occur in those antigens having cysteine residues.

G. Diagnostic Systems

A diagnostic system in kit form of the present invention includes, in an amount sufficient for at least one assay, a composition comprising a HIV or HCV antigen of the current invention as a separately packaged reagent. Instructions for use of the packaged reagent are also typically included. “Instructions for use” typically include a tangible expression describing the reagent concentration or at least one assay method parameter such as the relative amounts of reagent and sample to be admixed, maintenance time periods for reagent/sample admixtures, temperature, buffer conditions and the like.

In preferred embodiments, the diagnostic system of the present invention further includes a label or indicating means capable of signaling the formation of a complex containing a recombinant antigen. As used herein, the terms “label” and “indicating means” in their various grammatical forms refer to single atoms and molecules that are either directly or indirectly involved in the production of a detectable signal to indicate the presence of a complex. Any label or indicating means can be linked to or incorporated in an expressed protein or polypeptide, or used separately, and those atoms or molecules can be used alone or in conjunction with additional reagents. Such labels are themselves well-known in clinical diagnostic chemistry and constitute a part of this invention only insofar as they are utilized with otherwise novel proteins methods and/or systems.

The linking of labels, i.e., labeling of, polypeptides and proteins is well known in the art. For instance, antibody molecules produced by a hybridoma can be labeled by metabolic incorporation of radioisotope-containing amino acids provided as a component in the culture medium. See, for example, Galfre et al., Meth. Enzymol., 73:346 (1981). The techniques of protein conjugation or coupling through activated functional groups are particularly applicable. See, for example, Avrameas, et al., Scand. J. Immunol., Vol. 8 Suppl. 7:7-23 (1978), Rodwell et al., Biotech., 3:889-894 (1984), and U.S. Pat. No. 4,493,795.

The diagnostic systems can also include, preferably as a separate package, a specific binding agent. A “specific binding agent” is a molecular entity capable of selectively binding a reagent species of the present invention but is not itself a protein expression product of the present invention. Exemplary specific binding agents are antibody molecules, complement proteins or fragments thereof, protein A, immobilized metal ion chelates, immobilized glutathione and the like. Preferably the specific binding agent can bind the recombinant antigen when the antigen is present as part of a complex.

In preferred embodiments the specific binding agent is labeled. However, when the diagnostic system includes a specific binding agent that is not labeled, the agent is typically used as an amplifying means or reagent. In these embodiments, the labeled specific binding agent is capable of specifically binding the amplifying means when the amplifying means is bound to a reagent species-containing complex.

The diagnostic kits of the present invention can be used in an “ELISA” format to detect the presence or quantity of antibodies in a body fluid sample such as serum, plasma or saliva that react with any of the antigens of the present invention. “ELISA” refers to an enzyme-linked immunosorbent assay that employs an antibody or antigen bound to a solid phase and an enzyme-antigen or enzyme-antibody conjugate to detect and quantify the amount of an antigen or antibody present in a sample. A description of the ELISA technique is found in Chapter 22 of the 4th Edition of Basic and Clinical Immunology by D.P. Sites et al., published by Lange Medical Publications of Los Altos, Calif. in 1982 and in U.S. Pat. Nos. 3,654,090; 3,850,752; and 4,016,043, which are all incorporated herein by reference.

In preferred embodiments, an HIV or HCV antigen of the present invention can be affixed to or coated on a solid matrix to form a solid support that is separately packaged in the subject diagnostic systems. The antigen is typically affixed to the solid matrix by adsorption from an aqueous medium although other modes of affixation, well known to those skilled in the art can be used. Useful solid matrices are well known in the art. Such materials include the cross-linked dextran available under the trademark SEPHADEX from Pharmacia Fine Chemicals (Piscataway, N.J.); agarose; beads of polystyrene about I micron to about 5 millimeters in diameter available from Abbott Laboratories of North Chicago, Ill.; polyvinyl chloride, polystyrene, cross-linked polyacrylamide, nitrocellulose- or nylon-based webs such as sheets, strips or paddles; or tubes, plates or the wells of a microtiter plate such as those made from polystyrene or polyvinylchloride.

The HIV or HCV antigen, labeled specific binding agent or amplifying reagent of any diagnostic system described herein can be provided in solution, as a liquid dispersion or in a substantially dry format, e.g., in lyophilized form. Where the indicating means is an enzyme, the enzyme's substrate can also be provided in a separate package of a system. A solid support such as the before-described microtiter plate and one or more buffers can also be included as separately packaged elements in this diagnostic assay system.

The packages discussed herein in relation to diagnostic systems are those customarily utilized in diagnostic systems. Such packages include glass and plastic (e.g., polyethylene, polypropylene and polycarbonate) bottles, vials, plastic and plastic-foil laminated envelopes and the like.

### EXAMPLES

The examples illustrate the present invention but in no way limit its scope.

### Example 1

Isolation of the HIV p24 Gene and Construction of Expression Vector

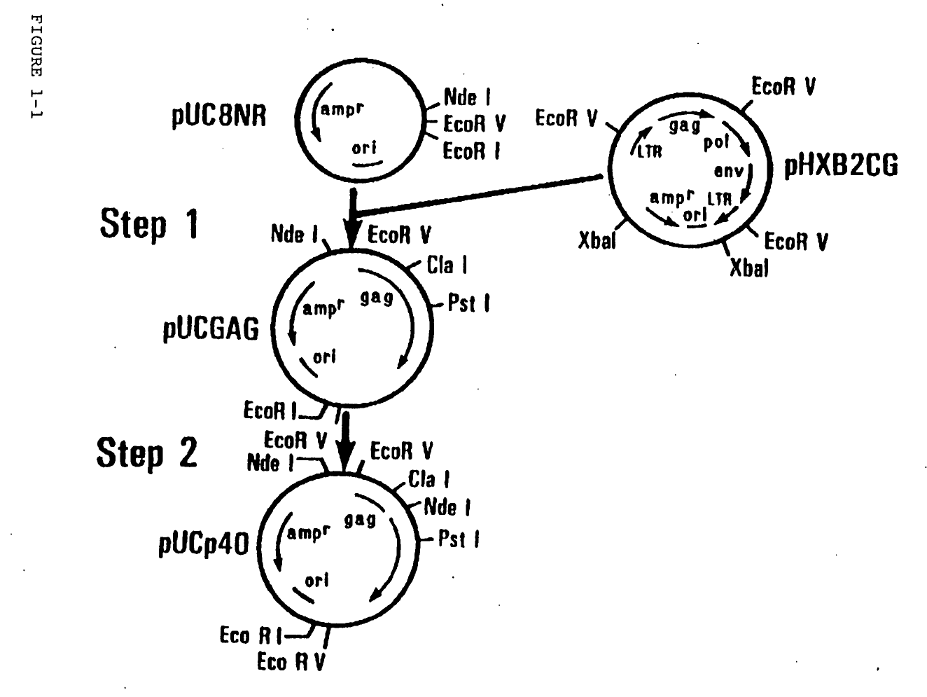

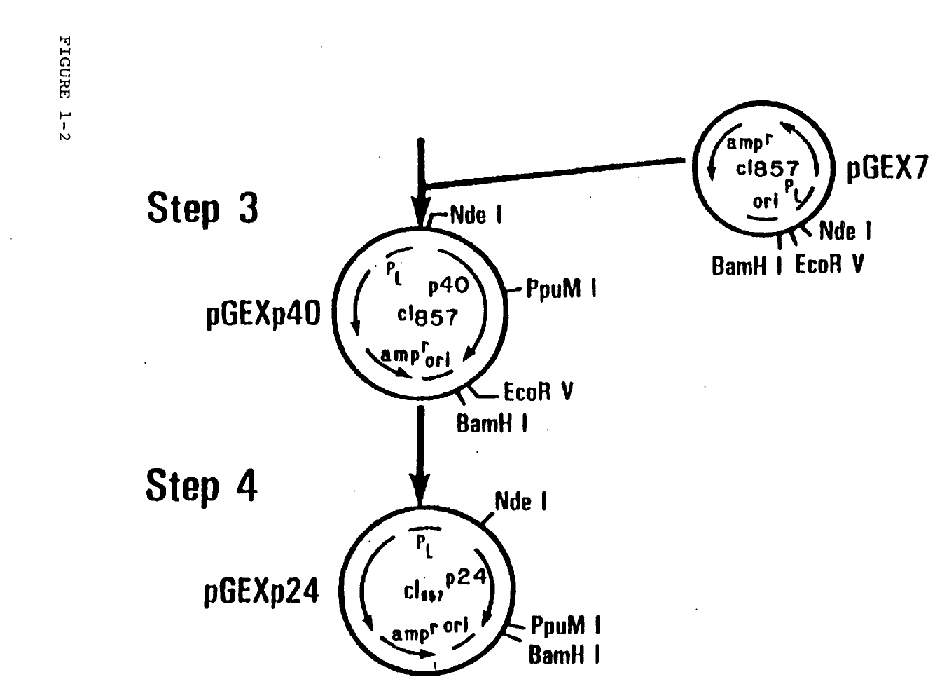

The gag region from the pHXB2CG plasmid clone of HTLV IIIB (obtained from Dr. Robert Gallo, National Cancer Institute, Bethesda, Md.) was isolated by EcoRV restriction enzyme digestion of plasmid pHXB2CG and the resulting 2.86 kilobase fragment was isolated and inserted by ligation into the EcoRV site of a modified pUC8 vector (pUC8NR) to form plasmid pUCGAG (FIG. 1, Step 1).

The plasmid (pUCGAG) was mutagenized to generate an ATG translational initiation codon and an NdeI restriction enzyme site (CATATG) at the beginning of the p24 structural gene by the following series of manipulations (FIG. 1, Step 2). After transformation of pUCGAG into the methylation deficient dam- strain of E. coli, New England Biolabs, a gap was created in the pUCGAG DNA at the p24 amino terminus by cutting with the ClaI and PstI restriction enzymes to form gapped pUCGAG that lacks the smaller DNA segment from the p24 amino terminus. Ten micrograms of gapped pUCGAG DNA and 10 micrograms of pUCGAG DNA cut with the restriction enzyme EcoRI were both subjected to electrophoresis on a 1% agarose gel, and the DNA fragments were each separately isolated from the agarose gel by electroelution (Model 1750 sample concentrator; ISCO, Lincoln, Nebr.), combined, extracted twice with a 50/50 mixture of phenol and chloroform, and precipitated with the addition of sodium acetate (final concentration, 100 mM) and three volumes of ethanol.

The precipitated DNAs were collected by centrifugation and resuspended to a concentration of 25 micrograms per milliliter in water. After addition of an equal volume of annealing buffer (80% formamide, 100 mM Tris, pH 8.0, 25 mM EDTA) the resuspended DNAs were denatured by boiling for 5 minutes and allowed to anneal at 37° C. for 30 minutes. The annealed DNAs were diluted with an equal volume of water and precipitated in ethanol as described above to form precipitated annealed DNA.

The Ndel and ATG sequences were joined to the amino terminus of the p24 gene using the following synthetic oligonucleotide:

5′-CCAAAATTACCATATGCCAATCGTGCAGAAC-3′ (SEQ ID NO:19)

The 10 nucleotides at the 5′ end and 9 nucleotides at the 3′ end of this oligonucleotide are homologous to the HTLV IIIB DNA sequence (University of Wisconsin genetic database). The intervening nucleotides were chosen to minimize the formation of secondary structures within the oligonucleotide and within the RNA expected to be generated from this sequence during expression of these sequences in E. coli.

Forty picomoles of the above oligonucleotide (synthesized on a Pharmacia Gene Assembler) was phosphorylated (as described in Molecular Cloning by T. Maniatis, E. F. Fritsch and J. Sambrook, Cold Spring Harbor Laboratory, 1982, p.125) and admixed with 2.5 micrograms of the precipitated annealed DNA described above. The admixed DNAs were then annealed by heating the admixture to 65° C. for 5 minutes and then cooling to room temperature over the course of an hour in ligase buffer (op. cit., p.474). The resulting DNA molecule (i.e., a gapped template) containing the precipitated annealed DNA described above and the gapped template with the annealed oligonucleotide was then repaired in vitro in ligase buffer by incubating for 3 hours at 15° C. in the presence of 25 μM of each deoxynucleoside triphosphate, 50 μM adenosine triphosphate, 5 units of T4 DNA ligase and 1 unit of the Klenow fragment of E. coil DNA polymerase.

After transformation into competent cells of the JM83 strain of E. coli the bacterial colonies were screened by hybridization with radiolabelled oligonucleotide on nitrocellulose (op. cit., pp.250-251, 313-329). A single colony was isolated by this procedure containing the plasmid pUCp40 (FIG. 1), with the DNA sequence for the amino terminal sequence of the p24 gene as disclosed in U.S. Pat. No. 5,470,720.

The DNA fragment from pUCp40 encoding a p24-p5 fusion protein referred to as p40 below and located between the NdeI restriction enzyme site created by the above mutagenesis and the EcoRV site, was isolated by digesting plasmid pUCp40 with NdeI and EcoRV followed by separation on an agarose gel, extraction and precipitation of the separated fragment.

Plasmid pGEX7 DNA was linearized by digestion with NdeI and EcoRV. Plasmid pGEX7 is a bacterial expression vector deposited as plasmid PHAGE 38 with the American Type Culture Collection (ATCC) on Jun. 9, 1988 and given the ATCC accession number 40464. It contains a lambda bacteriophage promoter (P_{L}), the gene for its temperature sensitive repressor (cI857), the sequence AGGAAGGGTTTTTCAT and an origin of replication (ori).

The digestion of pGEX7 with NdeI and EcoRV results in the production of two linear fragments, one of which contains the ampr and cI857 genes and the origin of replication and has NdeI and EcoRV cohesive termini. The above described p40 gene-containing NdeI/EcoRV restriction fragment of pUCp40 was then ligated to the pGEX7 NdeI/EcoRV amp^{r} gene-containing fragment via their respective NdeI and EcoRV termini to form the plasmid pGEXp40 (FIG. 1, Step 3).

The sequences of pGEXp40 encoding p15 were removed from plasmid pGEXp40 by restriction digestion with the enzymes PpuMI and BamHI. Thereafter the 3′ end of the p24 gene was reconstructed as indicated by FIG. 1, Step 4 by synthesizing two complementary oligonucleotides (SEQ ID NO:20 and SEQ ID NO:21) which when annealed form a duplex comprising translational stop codons and overhanging ends corresponding to PpuMI and BamHI restriction enzyme sites. The resulting rDNA plasmid, pGEXp24, expresses an HIV p24 antigen.

### Example 2

Formation of Composite DNAs Comprising the pGEXp24 Vector with an Inserted Gene for a Conserved Envelope gp41 (Subtype 0) Antigen.

The plasmid pGEXp24, was linearized by digestion with the restriction enzyme PpuMI and purified by phenol-chloroform extraction followed by precipitation with ethanol. Two complementary oligonucleotides (sequences given by nucleotides 686 to 763 and the complement of nucleotides 689 to 766 of SEQ ID NO: 1) forming protruding cohesive termini when annealed, were synthesized. The synthetic oligonucleotides were allowed to form a duplex by mixing and heating to 90° C. for a approximately 3 minutes, followed by annealing at room temperature for a period of 10 minutes. The hybrid molecule represents a hybrid gene sequence encoding the p24 molecule interrupted after codon 225 by a linker amino acid (lysine), envelope sequence (amino acids 227-249) for the conserved region of HIV Subtypte 0 gp41 polypeptide, strain ANT, followed by a repetition of p24 residues 224 and 225 and then p24 residues 226-232.

A similar hybrid oligonucleotide representing the gp41 conserved region of HIV Subtype 0, strain MVP 5180, was formed by synthesizing complementary oligonucleotides with the sequences given by nucleotides 686 to 763 and the complement of nucleotides 689 to 766 of SEQ ID NO:3.

A third hybrid oligonucleotide representing the gp41 conserved region of HIV Subtype 0, strain GenBank X84328 was formed by synthesizing complementary oligonucleotides with the sequences given by nucleotides 686 to 763 and the complement of nucleotides 689 to 766 of SEQ ID NO:5.

All three duplexes were separately mixed with the linearized pGEXp24 vector and 400 U of T4 ligase and incubated in ligase buffer containing I mM ATP at 16° C. overnight. Subsequent transformation into competent E. coli and screening of mini-preparations by AvaII digestion allowed for the selection of clones containing the insert as described in U.S. Pat. No. 5,470,720. Mini-inductions confirmed high level synthesis of the gene product of interest, as evidenced by lysing induced cultures in the presence of SDS and running the lysate on a 16% SDS PAGE. The plasmid containing the hybrid gene formed by the first oligonucleotide pair, designated pGEXp24gp41-ANT, comprises the nucleotide sequence given by SEQ ID NO: 1. The plasmid containing the hybrid gene formed by the second oligonucleotide pair, designated pGEXp24gp4-MVP, comprises the nucleotide sequence given by SEQ ID NO:3. The plasmid containing the hybrid gene formed by the third oligonucleotide pair, designated pGEXp24gp41-X84328, comprises the nucleotide sequence given by SEQ ID NO:5.

### Example 3

Purification of Recombinant p24-gp41 (Subtype 0) Fusion Proteins

Plasmids containing the lambda promoter (pL) are normally carried in a strain of bacteria containing a lysogen of bacteriophage lambda in order to minimize the expression of the gene product of interest during the manipulation of DNAs. The pGEX7-based plasmids described in Example 1 were all carried in a lysogen of the MM294 strain of E. coli. Expression from the lambda promoter of pGEX7 can be demonstrated by transfer of the plasmid into an uninfected bacterial host (e.g., E. coli strain W3110, accession no. #27325, ATCC, Rockville, Md.) and inactivation of the cI repressor protein at 42° C. Competent E. coli (strain W3110, 100 ml bacterial suspension) were transformed with 1 μl of pGEXp24gp41-ANT, pGEXp24gp41-MVP or pGEXp24gp41-X84328. After 60 minutes on ice, the bacteria were diluted to 1 ml with LB medium and incubated for a further 60 minutes at 30° C. Aliquots of the culture were than plated on ampicillin containing agar plates which were held at 30° C. for at least 24 hours. A colony was picked and inoculated into 5 ml of LB medium and incubated for approximately 6 hours at 30° C. 1 ml of the growing culture, indicated by developing turbidity of the inoculum, was then transferred to a 1 liter flask for further overnight culture, using a temperature controlled shaker at 300 rpm. The main culture was initiated the following morning by inoculating each of 6 flasks containing 0.9 liter of LB Medium and 50 mg ampicillin/liter with 100 ml of the overnight culture. The flasks were shaken at 350 rpm for 1.5 hours. The cultures were induced by raising the temperature to 42° C. and maintained at that temperature for 4 hours. The cells were harvested by centrifugation (Sorvall, GSA Rotor, 7,000 rpm, 10 minutes in the cold), transferred to a storage container and typically stored frozen until used for purification.

The cell paste from 6 liter cultures (approximately 30 g of frozen bacteria) were thawed and suspended in an equal volume of 0.2 M phosphate buffer, pH 7.0, containing 10 mM EDTA and 10 mM benzamidine. Lysozyme (1 mg/g cell paste) and PMSF (0.2 mg/g cell paste) was added and the suspension stirred for approximately 30 minutes at room temperature. During this period, the material became very viscous. The cells were then placed in an ice bath and subjected to 3 minutes of sonication on ice with intervening cooling periods of 1-2 minutes.

Soluble materials were removed by centrifigation (Sorvall, SS-34 rotor, 20,000 rpm for 30 minutes) and the extraction procedure was repeated using 0.2 M phosphate buffer containing 10 mM EDTA and 10 mM benzamidine. The combined supernatants were discarded and the sediment suspended in 6 M urea containing 0.02 M Tris-HCl buffer, pH 8.6. The suspension was subjected to a further cycle of sonication on ice (60 seconds) and the centrifigation was repeated. The supernatant was saved and the sediment re-extracted once, using urea-tris buffer of the same composition. The combined supernatants were treated with ammonium sulfate (0.3 g/ml of solution), kept at 4° C. for about 30 minutes and then centrifuged as described above. A large precipitate had formed which was dissolved in approximately 20 ml of 6 M Guanidine-HCl, containing 0.1 M phosphate buffer, 5 mM EDTA, pH 7.0. The solubilized material was clarified by renewed centrifugation and then applied to a 5×105 cm column, containing Sepharose S-300 gel and equilibrated with 6 M Guanidine-HCl in 0.1 M phosphate-5 mM EDTA buffer, pH 7.0. Fractions (10 ml) were eluted and, following dialysis against 6 M urea of selected aliquots, analyzed by SDS gel electrophoresis. Based on the gel pattern, appropriate fractions containing gene products migrating to a position of the gel which corresponded to that reference proteins, or, if such was unavailable, similar to the band appearing as a consequence of the induction of cultures carrying the expression vector, were pooled and exhaustively dialyzed against 4 M urea containing 0.015 M Tris-HCl buffer, pH 8.6.

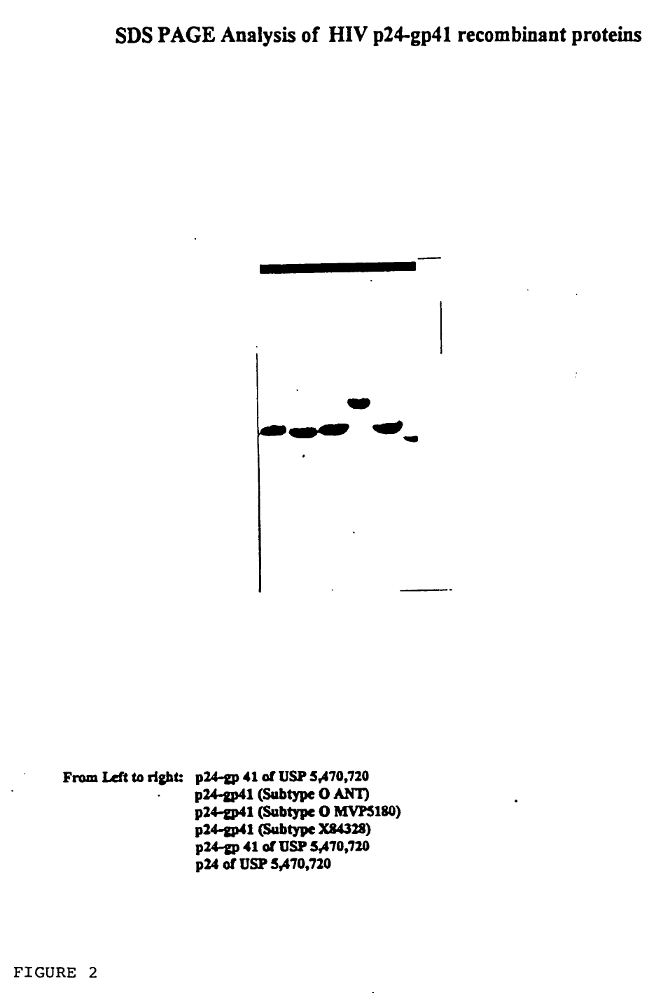

The dialyzed, clear solution was applied to a column (2.5×30 cm) of DEAE-Sepharose equilibrated with 4 M urea-0.015 M Tris-HCl buffer, pH 8.6. Following application of the sample and washing to remove non-bound constituents, the protein of interest was eluted with a salt gradient (250×250 ml, 0-0.1 M NaCl in the initial Tris-HCl buffer containing 4 M urea) and monitored by analysis in 16% SDS PAGE. Fractions containing the protein of interest were pooled and adjusted to pH 5.6 by addition of glacial acetic acid. The pH-adjusted pooled material was then applied to a column (2.5×20 cm) of CM Sepharose equilibrated with 20 mM sodium acetate buffer, pH 5.6 containing 4 M urea. A salt gradient (250×250 ml, 0-0.4M NaCl in the same urea-containing acetate buffer) was applied and fractions were collected. Fractions were again analyzed for the protein of interest. These fractions containing purified protein were pooled and stored at frozen at −20° C. FIG. 2 shows an analytical SDS gel of the three recombinant p24-gp41 hybrid proteins of subtype O after being purified in accordance with the above protocol.

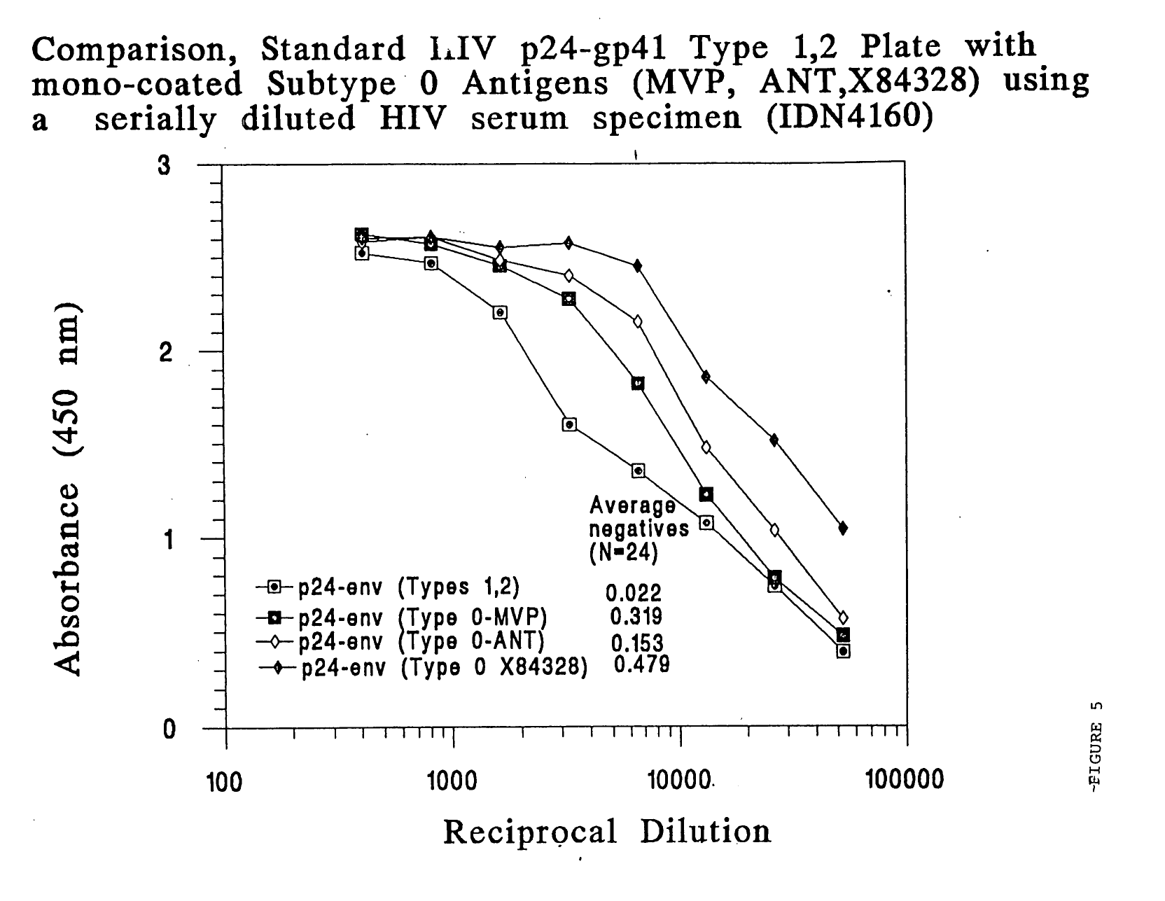

To test for immune reactivity with HIV positive sera, polystyrene wells (Nunc, Polysorp) were coated with mixtures of the p24-gp41 hybrid proteins described above in concentrations of 1 μg/ml for 16 hours at 4° C. After blocking with 3% bovine serum albumin overnight, the plates were dried under vacuum and then used to analyze the immune reactivity against sequential dilutions of a serum known to test positive for HIV antibody. FIG. 5 shows a titration curve using the three newly synthesized antigens in comparison with the prototype gene product obtained from pGEXp24-gp41 as disclosed in U.S. Pat. No. 5,470,720. The three antigens produce strong immune reactivity with this serum, comparable to that seen with the reference protein.

### Example 4

Formation of a Recombinant HCV Capsid Protein Gene Joined to pGEX7 for Synthesis of Carrier-free Polypeptide.

A. Isolation of HCV Clones and Sequence Analysis

(1) Isolation of HCV RNA and Preparation of cDNA

As a source for HCV virions, blood was collected from a chimpanzee infected with the Hutchinson (Hutch) strain exhibiting acute phase HCV. Plasma was clarified by centrifugation and filtration. Virions were then isolated from the clarified plasma by immunoaffinity chromatography on a column of HCV IgG (Hutch strain) coupled to protein G sepharose. HCV RNA was eluted from the sepharose beads by soaking in guanidinium thiocyanate and the eluted RNA was then concentrated through a cesium chloride (CsCl) cushion. Maniatis et al., Molecular Cloning: A Laboratory Manual, Maniatis et al., eds. Cold Spring Harbor, New York (1989).

The purified HCV RNA was used as a template in a primer extension reaction admixture containing random and oligo dT primers, dNTP's, and reverse transcriptase to form first strand cDNAs. The resultant first strand cDNAs were used as templates for synthesis of second strand cDNAs in a reaction admixture containing DNA polymerase I and RNAse H to form double stranded (ds) cDNAs (Maniatis et al., supra). The synthesized ds cDNAs were amplified using an asymmetric synthetic primer-adaptor system wherein sense and anti-sense primers were annealed to each other and ligated to the ends of the double stranded HCV cDNAs with T4 ligase under blunt-end conditions to form cDNA-adaptor molecules. Polymerase chain reaction (PCR) amplification was performed by admixing the cDNA-adaptor molecules with the same positive sense adaptor primers, dNTP's and TAQ polymerase to prepare amplified HCV cDNAs. The resultant amplified HCV cDNA sequences were then used as templates for subsequent amplification in a PCR reaction with specific HCV oligonucleotide primers.

(2) Synthesis of Oligonucleotides For Use in HCV Cloning

Oligonucleotides were selected to correspond to the 5′ sequence of Hepatitis C virus which encodes the HCV structural capsid and envelope proteins (HCJ1 sequence: Okamoto et al., Jap. J. Exp. Med., 60:167-177, 1990). The selected oligonucleotides were synthesized on a Pharmacia Gene Assembler according to the manufacturer's instruction, purified by polyacrylamide gel electrophoresis.

(3) PCR Amplification of HCV cDNA

PCR amplification was performed by admixing the primer-adapted amplified cDNA sequences prepared in Example 4.A.(1) with the synthetic oligonucleotide primer pair 690:694. (690: nucleotides 16-36 of SEQ ID NO:9; 694: complement of nucleotides 162-178 of SEQ ID NO:9). The resulting PCR reaction admixture contained the primer-adapted amplified cDNA template, oligonucleotides 690 and 694, dNTP's, salts (KC1 and MgCl₂) and TAQ polyrnerase. PCR amplification of the cDNA was conducted by maintaining the admixture at a 37° C. annealing temperature for 30 cycles. Aliquots of samples from the first round of amplification were reamplified at a 55° C. annealing temperature for 30 cycles under similar conditions.

(4) Preparation of Vectors Containing PCR Amplified ds DNA

Aliquots from the second round of PCR amplification were subjected to electrophoresis on a 5% acrylamide gel. After separation of the PCR reaction products, the region of the gel containing DNA fragments corresponding to the expected 690:694 amplified product of approximately 224 bp was excised and purified following standard electroelution techniques (Maniatis et al., supra). The purified fragments were kinased and cloned into the pUC18 plasmid cloning vector at the SmaI polylinker site to form a plasmid containing the DNA segment 690:694 joined to pUC18.

The resulting mixture containing pUC18 and a DNA segment corresponding to the 690:694 sequence region was then transformed into the E. coli strain JM83. Plasmids containing inserts were identified as lac- (white) colonies on X-gal medium containing ampicillin. pUC18 plasmids which contained the 690:694 DNA segment were identified by restriction enzyme analysis and subsequent electrophoresis on agarose gels, and were designated pUC18 690:694.

(5) Sequencing of HCV Clones that Encode the Putative Capsid Protein

Two independent colonies believed to contain a pUC18 vector having the HCV Hutch strain 690:694 DNA segment (pUC18-690:694) that codes for the amino terminus of the capsid protein were amplified and used to prepare plasmid DNA by CsCl density gradient centrifugation by standard procedures (Maniatis et al., supra). The plasmids were sequenced using ³⁵S dideoxy procedures with pUC18 specific primers. The two plasmids were independently sequenced on both DNA strands to assure the accuracy of the sequence.

(6) Preparation of HCV Clones from the 5′ End of the Genome

To obtain a clone encoding the remainder of the of the HCV Hutch capsid region (Okamoto et al., supra), the oligonucleotide pair 693:691 (693: nucleotides 162-178 of SEQ ID NO:9; 691: complement of nucleotides 355-375 of SEQ ID NO:9) were used in PCR reactions. cDNA was prepared as described in Example 4.A.(1) from viral HCV RNA (Hutch) and used in PCR amplification as described in Example 4.A.(3) with the oligonucleotide pair 693:691. The resultant PCR amplified ds DNA was then cloned into pUC18 cloning vectors and screened for inserts as described in Example 4.A.(4) to form pUC18-693:691. Clones were then sequenced with pUC18 specific primers as described in Example 4.A.(5). Plasmid pUC18-693:691 was found to contain a HCV DNA segment that is 157 bp in length and corresponds to the HCV prototype HJC1 sequence (SEQ ID NO:9) from nucleotides 218-375.

B. Production of Recombinant DNA (rDNA) Encoding Fusion Proteins

(1) Introduction of the 690:694 Fragment into pGEX-3X for Expression of GST Fusion Protein

The pUC18-690:694 DNA was subjected to restriction enzyme digestion with EcoRI and BamHI to release a DNA segment containing the HCV 690:694 fragment. The released DNA segment was subjected to acrylamide electrophoresis and a DNA segment containing the 224 bp HCV insert plus portions of the pUC18 polylinker was then excised and eluted from the gel as described in Example 4.A.(4). The DNA segment was extracted with a mixture of phenol and chloroform, and precipitated.

The precipitated DNA segment was resuspended to a concentration of 25 μg/ml in water and treated with the Klenow fragment of DNA polymerase to fill in the staggered ends created by the restriction digestion. The resultant blunt-ended 690:694 containing segment was admixed with the bacterial expression vector pGEX-3X, (Pharmacia Inc., Piscataway, N.J.) which was linearized with the blunt end restriction enzyme SmaI. The admixed DNAs were then ligated by maintaining the admixture overnight at 16° C. in the presence of ligase buffer and 5 units of T4 DNA ligase to form a plasmid of 690:694 DNA segment joined to pGEX-3X.

(2) Selection and Verification of Correct Orientation of Ligated Insert

The ligation mixture containing the pGEX-3X vector and the 690:694 DNA containing segment was transformed into host E. coli strain W3110. Plasmids containing inserts were identified by selection of host bacteria containing vector in Luria broth (LB) media containing ampicillin. Bacterial cultures at stationary phase were subjected to alkaline lysis protocols to form a crude DNA preparation. To screen for a vector containing the 690:694 DNA segment, plasmid DNA was digested with the restriction enzyme Xhol, which cleaves within the 690:694 DNA segment, but not within the pGEX-3X vector.

Several 690:694 DNA segment-containing vectors were amplified and the resultant amplified vector DNA was purified by CsCl density gradient centrifuigation. The DNA was sequenced across the inserted DNA segment ligation junctions by ³⁵S dideoxy methods with a primer which hybridized to the pGEX-3X. Vectors containing 690:694 DNA segment having the correct coding sequence for in-frame translation of an HCV structural protein were thus identified and selected to form pGEX-3X-690:694.

(3) Structure of the Fusion Protein

The pGEX-3X vector is constructed to allow for inserts to be placed at the C terminus of Sj26, a 26-kDa glutathione-S-transferase (GST; EC2.5.1.18) encoded by the parasitic helminth Schistosoma japonicum. The insertion of the 690:694 HCV fragment in-frame behind Sj26 allows for the synthesis of the Sj26-HCV fusion polypeptide. The HCV polypeptide can be cleaved from the GST carrier by digestion with the site-specific protease factor Xa (Smith et al., Gene 67:31-40, 1988).

The resulting rDNA molecule, pGEX-3X-690:694, encodes an HCV fusion protein having an amino terminal polypeptide portion corresponding to residues 1 to 221 of GST, a four residue intermediate portion defining a cleavage site for the protease Factor Xa, a nine residue linker, a polypeptide portion corresponding to amino acid residue sequence 1 to 74 of SEQ ID NO:9 and a six residue tail.

(4) Introduction of the 690:694 Fragment into pGEX-3X

Plasmid pGEX-3X-693:691 was formed by first subjecting the plasmid pUC18-693:691 prepared in Example 4.A.(6) to restriction enzyme digestion with EcoRI and BamHI as in Example 4.B.(1). The purified DNA segment was admixed with and ligated to the pGEX-3X vector which was linearized by restriction enzyme digestion with EcoRI and BamBI in the presence of T4 ligase at 16° C. to form the plasmid pGEX-3X-693:691.

A pGEX-3X plasmid containing a 693:691 DNA segment was identified as in Example 4.B.(2) with the exception that crude DNA preparations were digested with EcoRI and BamBI to release the 693:691 insert. A pGEX-3X vector containing a 693:691 DNA segment having the correct coding sequence for in-frame translation of an HCV structural protein was identified by sequence analysis as performed in Example 4.B.(2) and selected to form pGEX-3X-693:691.

The resulting vector encodes a fusion protein (GST:HCV 693:691) that is comprised of an amino-terminal polypeptide portion corresponding to residues 1-221 of GST, an intermediate polypeptide portion corresponding to residues 222-225 and defining a cleavage site for the protease Factor Xa, a five residue linker portion, a carboxy-terminal polypeptide portion corresponding to amino acid residues 69 to 120 of the HCV capsid antigen, and a three residue tail.

C. Plasmids Encoding Complete Capsid Proteins

(1) Construction of a Vector Expressing a Composite Gene

To generate a composite gene spanning the entire amino acid region of 1-120 and to create an operative linkage of the gene to the first DNA segment of this invention,(i.e., AGGAGGGTTTTTCAT), the following experiments were conducted. The above described plasmids pGEX-3X-690:694 and pGEX-3X-691 :693, containing base pairs 1-224 and 203-360, respectively, of an HCV capsid gene (U.S. Ser. No. 07/573,643) were used as target templates for each of two separate PCR reactions encompassing the following primer pairs.

A first PCR reaction was performed using a primer pair with sequences given by SEQ ID NO:22 and the complement of nucleotides 219-239 of SEQ ID NO:7 to amplify a 210 base pair fragment from plasmid pGEX-3X-690:694. The amplified fragment contains a single NdeI and EagI site at the 5′ and 3′ ends, respectively.

A second PCR reaction was performed using a primer pair (sequences given by SEQ ID NO:23 and nucleotides 219 to 239 of SEQ ID NO:7) to amplify a 150 bp fragment from plasmid pGEX-3X-691:693. The second amplified fragment contains an EagI site at the 5′ end and an EcoRI site at the 3′ of the amplimer.

The PCR products were cut with the NdeI and EagI (first PCR reaction product) and with EagI and EcoRI (second PCR reaction product). In a third digestion, the pGEX7 vector was digested with NdeI and EcoRI. Following isolation by preparative electrophoresis in 5% acrylamide of each DNA segment, a three-way ligation mixture containing the isolated and restricted PCR reaction products and isolated pGEX7 vector was formed, and allowed to incubate with T4 Ligase overnight at 16° C. The mixture was then transformed into competent cells, colonies were selected for plasmid mini-preparations and subsequently analyzed by redigestion with NdeI and EcoRI. The vector pGEX-C120H-V68 released an insert of the proper length upon restriction digestion with NdeI and EcoRI and had the nucleotide sequence shown in SEQ ID NO: 7. Compared with the sequence for the HUTCH strain, pGEX-C120H-V68 has amino acid substitutions at amino acid 4 (Ile instead of Asn) and amino acid 68 (Val instead of ala) shown in SEQ ID NO: 8.

(2) Vectors Expressing Modified Capsid Proteins

The codon at position 68 is included in a stretch of the DNA molecule spanned by two StyI sites, (nucleotides 212 and 259 of SEQ ID NO:7 are the first base in the StyI recognition sites). A plasmid vector containing the HUTCH sequence in this StyI fragment is made by ligating a DNA fragment formed by annealing complementary synthetic oligonucleotides with sequences given by nucleotides 213 to 259 and the complement of nucleotides 217 to 263 of SEQ ID NO: 9 into the StyI-digested pGEX-C120H-V68 vector. The proper orientation of the inserted DNA fragment is assured as the two StyI cohesive ends are different. The sequence of the resulting vector, pGEX-C120H, codes for alanine at amino acid 68 of the capsid sequence (SEQ ID NO: 10).

Alternative modifications of the capsid structure which substitute specific sequences from other genotypes of HCV may be accomplished by the similar use of other synthetic oligonucleotide pairs with StyI/StyI cohesive ends. For example, an amino acid sequence corresponding to the HCV capsid of genotype 2 may be substituted by annealing a synthetic oligonucleotide pair with the sequences given by nucleotides 213 to 259 and the complement of nucleotides 217 to 263 of SEQ ID NO: 11 and inserting the duplex into the StyIStyI region. The capsid encoded by the resulting pGEX-C120H-IS02 is given in SEQ ID NO:12. Plasmid pGEX-C120H-IS03 encoding particular amino acids corresponding to an HCV capsid protein of genotype 3 (SEQ ID NO: 14 is similarly obtained with the synthetic sequences given by nucleotides 213 to 259 and the complement of nucleotides 217 to 263 of SEQ ID NO:13.

### Example 5

Preparation of Purified HCV 1-120 Capsid Proteins

A. Transformation and Growth of Bacteria

Competent E. coli (strain W3110, 100 ul bacterial suspension) were transformed with 1 ul of purified pGEX-C120H-V68 plasmid containing the insert shown in SEQ ID NO:7. After 60 minutes on ice, the bacteria were diluted to 1 ml with LB medium and incubated for a further 60 minutes at 30° C. Aliquots of the culture were than plated on Amp-containing agar plates which were incubated at 30° C. for at least 24 hours. A colony was picked and inoculated into 5 ml of LB medium. After approximately 6 hours at 30° C., 1 ml of the growing culture, indicated by developing turbidity of the inoculum, was then transferred to a 1 liter flask for further overnight sub-culturing, using a temperature controlled shaker at 300 rpm. The main culture was initiated the following morning by inoculating each of 6 flasks containing 0.9 liter of LB and 50 mg ampicillin/liter with 100 ml of the overnight culture. The flasks were shaken at 350 rpm for 2 hours and the cultures were then induced by raising the temperature to 42° C. for 4 hours. The cells were harvested by centrifugation and typically stored frozen until used for purification.

B. Isolation of HCV Capsid Protein from Induced Cultures.

The cell paste from 6 liter cultures (approximately 30 g of frozen bacteria) was thawed and suspended in an equal volume of 0.2 M phosphate buffer, pH 7.0, containing 10 mM EDTA and 10 mM benzamidine. Lysozyme (1 mg/g cell paste) and PMSF (0.2 mg/g cell paste) were added and the suspension stirred for approximately 30 minutes at room temperature. During this period, the material became very viscous. The cells were then placed in an ice bath and subjected to 3 minutes of sonication on ice with intervening cooling periods of 1-2 minutes. Soluble materials were removed by centrifugation (Sorvall, SS-34 rotor, 20,000 rpm for 30 minutes) and the extraction procedure was repeated using 0.2 M phosphate buffer containing 10 mM EDTA and 10 mM benzamidine. The combined supernatants were discarded and the sediment suspended in 0.02 M Tris-HCl buffer, pH 8.6, containing 6 M urea. The suspension was subjected to a further cycle of sonication on ice (60 seconds) and the centrifugation was repeated. The supernatant was saved and the sediment re-extracted once, using urea-tris buffer of the same composition. The combined supernatants were treated with ammonium sulfate (0.3 g/ml of solution), kept at 4° C. for about 30 minutes and then centrifuged as described above. A large precipitate had formed which was dissolved in approximately 20 ml of 0.1 M phosphate buffer, pH 7.0, containing 5 mM EDTA and 6 M guanidine-HCl. The solubilized material was clarified by renewed centrifugation and then applied to a 5×105 cm column, containing Sepharose S-300 gel and equilibrated with the same buffer. Fractions (10 ml) were eluted and, following dialysis against 6 M urea of selected aliquots, analyzed by SDS gel electrophoresis. Based on the gel pattern, appropriate fractions were pooled and exhaustively dialyzed against 4 M urea containing 0.1 M sodium acetate buffer, pH 5.4. The dialyzed, clear solution was applied to a column (2.5×20 cm) of CM-Sepharose equilibrated with 4 M urea-0.1 M acetate buffer, pH 5.4.

Following application of the sample and washing to remove non-bound constituents, the protein of interest was eluted with a salt gradient (250×250 ml, 0-0.4 M NaCl in the initial urea-containing acetate buffer) and monitored by analysis of selected fractions by 16% SDS PAGE. Fractions containing pure protein were pooled and stored at frozen at −20° C. FIG. 3 shows an analytical SDS gel of purified capsid protein after being subjected to the procedure described.

### Example 6

Formation of a Fusion Protein Comprising GST and Amino Acids 2140 of the HCV Capsid Protein

A. Construction of Plasmids Encoding GST-Capsid Fusion Proteins

(1) Construction of a Hybrid Gene in pGEX-2T-CAP-B

Oligonucleotides 21-40(+) and 21-40(−) for constructing the vector pGEX-2T-CAP-B for expressing the CAP-B fusion protein were prepared as described in Example 4.A.(2) having nucleotide base sequences corrresponding to SEQ ID NO:24 and SEQ ID NO:25, respectively.

Oligonucleotides 21-40 (+) and 21-40 (−) were admixed in equal amounts with the pGEX-2T expression vector (Pharmacia) that had been predigested with EcoRI and BamHI and maintained under annealing conditions to allow hybridization of the complementary oligonucleotides and to allow the cohesive termini of the resulting double-stranded oligonucleotide product to hybridize with pGEX-2T at the EcoRI and BamHI cohesive termini. After ligation the resulting plasmid, designated pGEX-2T-CAP-B contains a single copy of the double-stranded oligonucleotide product and contains a structural gene coding for a fusion protein designated CAP-B, having an amino acid residue sequence shown in SEQ ID NO: 18 from residue 1 to residue 252.

(2) Insertion of Hybrid Gene into pGEX7-CAP-B1 for High Level Expression

A PCR reaction is performed using the primer pair with sequences given by SEQ ID NO:26 and SEQ ID NO:27 to amplify a 759 base pair fragment from plasmid pGEX-2T-CAP-B. The amplified fragment will contain a single NdeI and EcoRI site at the 5′ and 3′ ends, respectively.

The PCR product is cut with the NdeI and EcoRI. In a second digestion, the pGEX7 vector is separately digested with NdeI and EcoRI. Following isolation by preparative electrophoresis in 5% acrylamide of each DNA segment, a ligation mixture containing the isolated and restricted PCR reaction product and pGEX7 vector is formed, and incubated with T4 Ligase overnight at 16° C. The mixture is then transformed into competent cells. Colonies are selected for plasmid mini-preparations which can subsequently be analyzed by redigestion with NdeI and EcoRI. The resulting sequence is shown in SEQ ID NO: 17.

B. Structure of the Expressed CAP-B1 Protein

The fusion protein expressed by pGEX7-CAP-B is comprised of an amino-terminal polypeptide portion corresponding to residues 1-220 of glutathione-S-transferase, an intermediate polypeptide portion corresponding to residues 221-226 and defining a cleavage site for Thrombin, and a polypeptide portion corresponding to residues 227-246 defining a portion of the HCV capsid antigen that has the amino acid residue sequence 21-40 in SEQ ID NO: 10. CAP-B1 is identical to CAP-B except that it lacks the 6 amino acid residue tail following the residues that correspond to amino acids 21-40 of the HCV capsid.

### Example 7

Formation of Recombinant Carrier Free HCV Non-structural Antigen 794.

A. Construction of Plasmid Comprising Gene for 794 Antigen Joined to pGEX7

The gene for the nonstructural 794 antigen was prepared from clone 20 (Table 9 p. 109), the latter disclosed in PCT application PCT/US91/06037 and encompassing 105 amino acid codons of the NS3 region inserted into the SmaI site of the vector pUC18. The pUC18 vector containing the insert was redigested with SmaI and EcoRI and subsequently inserted into a similarly digested pGST-2T vector (GenBank Accession number XXU13850). This resulted in an expression vector producing a fusion protein with a contiguous GST-HCV NS3 fusion sequence, GST translation beginning at nucleotide 258 of the vector, the NS3 protein beginning at nucleotide 936. The NS3 gene was re-isolated from this vector by digesting with SmaI and EcoRI, which released a 330 base-pair fragment isolated by preparative electrophoresis.

The pGEX7 vector was modified as follows. A pair of complementary synthetic oligonucleotides with sequences given by SEQ ID NO:28 and SEQ ID NO:29, when annealed, form a duplex with protruding NdeI and BamHI cohesive ends. The duplex encodes 6 histidine residues as well as a SmaI and EcoRI restriction site, the latter followed by stop codons in all three reading frames. To insert the DNA segment into pGEX7, the vector was first digested with NdeI and BamHI and the intervening polylinker removed by electrophoresis. Ligation of the digested vector with the synthetic oligonucleotide was followed by transformation and analysis of several mini-preparations. The plasmids were screened for a SmaI restriction site which is present in the insert but not the parent vector. Of ten colonies screened, all showed the presence of the SmaI restriction site. A colony was picked and used for preparing a sufficient quantity of modified pGEX7 plasmid. The plasmid was then linearized by digesting with SmaI and EcoRI the vector fragment was separated from the small SmaI-EcoRI fragment. The digested modified pGEX7 vector was used for ligation with the gene for the nonstructural NS3 antigen.

Ligation of the digested modified pGEX7 vector and the SmaI-EcoRI fragment encompassing the gene for the NS3 antigen was carried out overnight in the presence of 400 U of T4 DNA ligase and 1 mM ATP. Transformation of the ligase mixture was followed by screening of mini-preparations which identified several clones that contained the inserted gene for the 794 antigen as indicated by electrophoresis in a 5% acrylamide gel. Several of these clones also expressed a protein of the expected molecular size in mini-inductions. One of the clones was selected for a 6 liter fermentation experiment. The fermentation/induction was carried as described in Example 5A.

B. Purification of 794 Antigen from Fermentation Broths

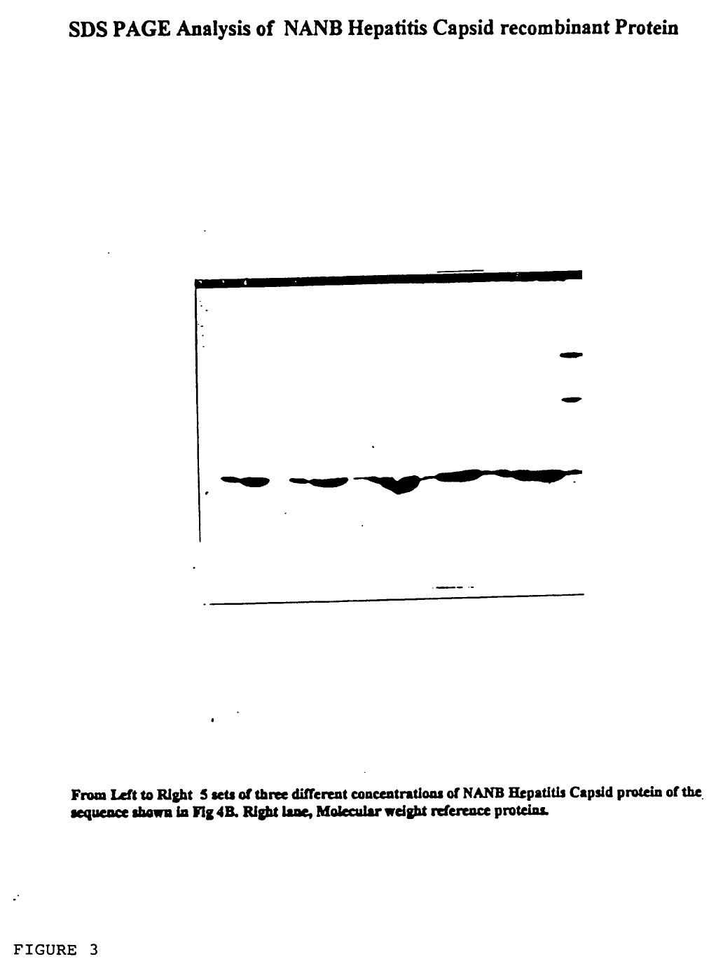

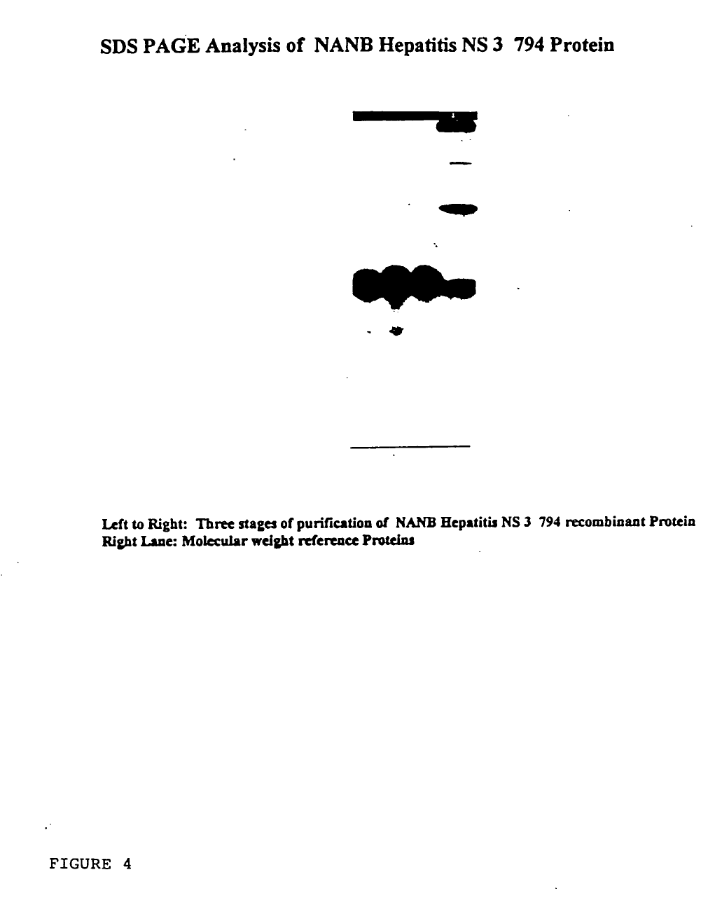

Frozen cell paste from induced cultures was thawed, suspended in buffer (0.2 M phosphate, 10 mM EDTA, 10 mM Benzamidine) and treated with lysozyme (1 mg/g cell paste) and PMSF (0.2 mg/g cell paste) followed by sonication as described in Example 5B. Following centrifugation, it was discovered that the protein of interest was directly soluble in the aqueous supernatant. Therefore, the sediment was discarded and the supernatant subjected to gel chromatography on a column (2.5×110 cm) of Sepharose S-300 eluted with 0.02 M Tris-HCl, pH 8.6, containing 0.2 M NaCl. Fractions were monitored with SDS PAGE and those containing the protein of interest pooled. The pooled material was subsequently applied in aliquots to a column (1×5 cm) of iminodiacetic acid derivatized Sepharose which had been previously charged with 50 mM nickel chloride and washed with 0.02 M Tris-HCl, 0.2 M NaCl. After absorption of the hexahistidine derivative of the NS3 794 antigen, it was eluted using successive elution steps with 0.03M Imidazole and 0.3 M Imidazole, respectively, in the above buffer. The protein emerged as a sharp peak with 0.3 M imidazole and was subsequently stored frozen at −20° C. An SDS PAGE analysis of the purified material is shown in FIG. 4.

### Example 8

Immune Reactivity of HCV Recombinant Antigens Expressed in pGEX7 Vectors.

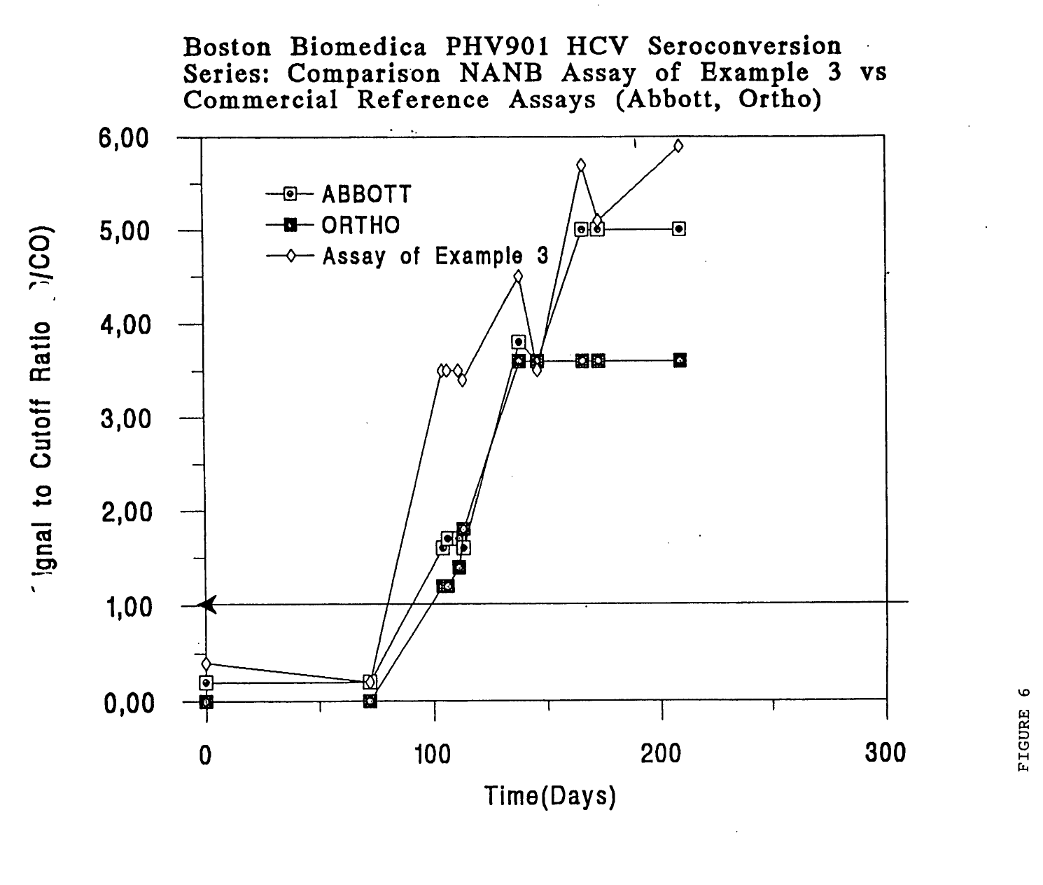

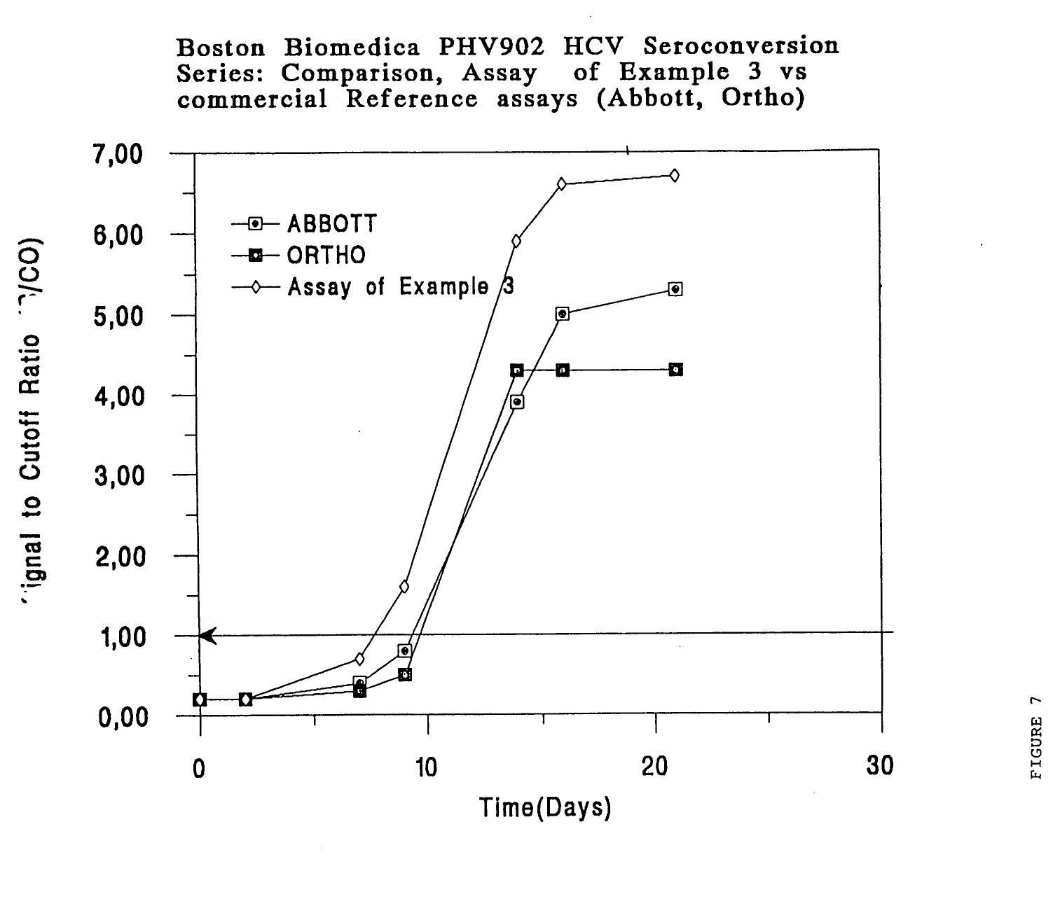

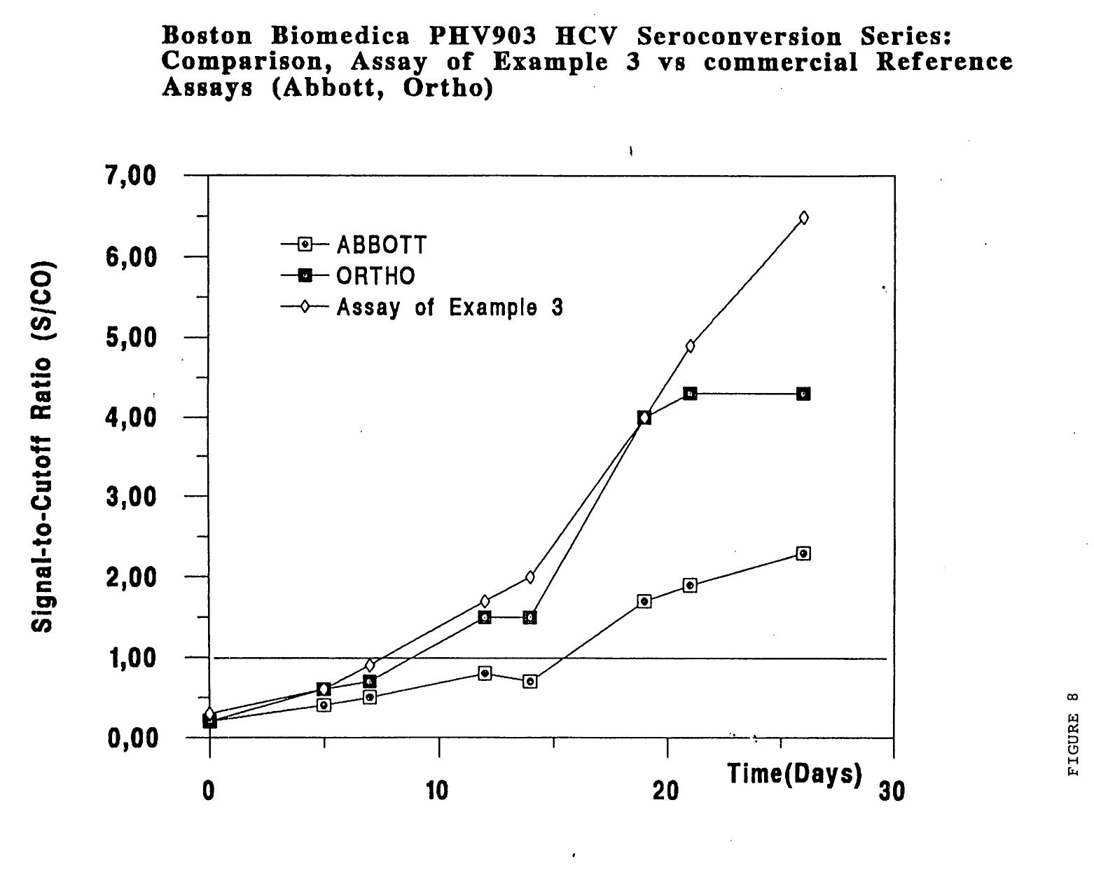

Polystyrene wells (Nunc, Polysorp) were coated with mixtures of the HCV capsid polypeptide (SEQ ID NO:8) in concentrations ranging between 1 and 4 μg/ml and the HCV 794 NS3 antigen (SEQ ID NO: 16) at 0.2-0.5 μg/ml. After blocking with 3% bovine serum albumin the plates were dried under vacuum and then used to analyze the immune reactivity against sera from individuals undergoing seroconversion and therefore known to develop antibody against HCV. The results are shown in FIGS. 6-8, each of which provide the signal to cut off values recorded for the assay using the source materials of the present invention and compared with the data from commercial immunoassays as supplied by the manufacturer of the conversion panels. These assays detected antibody at least as early, or earlier than the state-of-the art assays.

We claim:

1. A recombinant expression vector comprising a first nucleic acid having the sequence AGGAGGGTTTTTCAT operatively linked to a second nucleic acid comprising three domains, wherein said first domain has a nucleotide sequence which encodes amino acids 1-225 of an HIV p24 antigen, said second domain has a nucleotide sequence which encodes an HIV gp41 antigen or an antigenic fragment of said HIV gp4l antigen and said third domain has a nucleotide sequence which encodes amino acids 224 to 232 of an HIV p24 antigen.

2. The vector of claim 1, wherein said vector is pGEX7 comprising said first nucleic and second nucleic acids.

3. The vector of claim 1, wherein said first, second and third domains together encode amino acids 1-258 of SEQ ID NO:2.

4. The vector of claim 3, wherein said vector is pGEXp24gp41-ANT.

5. The vector of claim 1, wherein said first, second and third domains together encode amino acids 1-258 of SEQ ID NO:4.

6. The vector of claim 5, wherein said vector is pGEXp24gp41-MVP.

7. The vector of claim 1, wherein said first, second and third domains together encode amino acids 1-258 of SEQ ID NO:6.

8. The vector of claim 7, wherein said vector is pGEXp24gp41-X84328.

9. A procaryotic host cell comprising an expression vector of any one of claims 3, 5 or 7.

10. A method of producing an HIV p24-gp41 antigen, which comprises
(a) treating a host cell comprising an expression vector of any one of claims 3, 5 or 7 under conditions and for a time effective to express said antigen; and
(b) recovering said antigen. 11. A recombinant HIV p24-gp41 antigen produced by the method of claim 10.

12. A composition comprising the recombinant HIV p24-gp41 antigen of claim 11, wherein said composition is essentially free of procaryotic antigens and other HCV-related proteins.

13. A diagnostic system, in kit form, comprising, in an amount sufficient to perform at least one assay, the composition of an HIV p24-gp41 antigen according to claim 12.

14. The diagnostic system according to claim 13, wherein said HIV p24-gp41 antigen is affixed to a solid matrix.

15. A method of assaying a body fluid sample for the presence of antibodies against an HIV p24-gp41 antigen which comprises:
a) forming an immunoreaction admixture by admixing said body fluid sample with a composition of claim 12;
b) maintaining said immunoreaction admixture for a time period sufficient for any of said antibodies present to immunoreact with said antigen to form an immunoreaction product; and
c) detecting the presence of any of said immunoreaction product formed and thereby the presence of said antibodies.

16. The method of claim 15, wherein said detecting in step (c) comprises the steps of:
(i) admixing said immunoreaction product formed in step (c) with a labeled specific binding agent to form a labeling admixture, said labeled specific binding agent comprising a specific binding agent and a label;
(ii) maintaining said labeling admixture for a time period sufficient for any of said immunoreaction product present to bind with said labeled specific binding agent to form a labeled product; and
(iii) detecting the presence of any of said labeled product formed, and thereby the presence of said immunoreaction product.

17. The method of claim 16, wherein said specific binding agent is Protein A or at least one of the antibodies anti-human IgG and anti-human IgM.

18. The method of claim 16, wherein said label is a lanthanide chelate, biotin, an enzyme or radioactive isotope.

19. A recombinant expression vector comprising a first nucleic acid having the sequence AGGAGGGTTTTTCAT operatively linked to a second nucleic acid consisting of a nucleotide sequence which encodes amino acids 1-120 of an HCV capsid antigen.

20. The vector of claim 19, wherein said vector is pGEX7 comprising said first nucleic and second nucleic acids.

21. The vector of claim 19, wherein said amino acids are amino acids 1-120 of SEQ ID NO:8.

22. The vector of claim 21, wherein said vector is pGEX-C120H-V68.

23. The vector of claim 19, wherein said amino acids are amino acids 1-120 of SEQ ID NO: 10.

24. The vector of claim 23, wherein said vector is pGEX-C120H.

25. The vector of claim 19, wherein said amino acids are amino acids 1-120 of SEQ ID NO: 12.

26. The vector of claim 25, wherein said vector is pGEX-C120H-ISO2.

27. The vector of claim 19, wherein said amino acids are amino acids 1-120 of SEQ ID NO: 14.

28. The vector of claim 27, wherein said vector is pGEX-C120H-ISO3.

29. A procaryotic host cell comprising an expression vector of any one of claims 19, 21, 23, 25 or 27.

30. A method of producing an HCV capsid antigen consisting of amino acid residues 1-120 which comprises
(a) treating a host cell comprising an expression vector of any one of claims 19, 21, 23, 25 or 27 under conditions and for a time effective to express said antigen; and
(b) recovering said antigen.

31. A recombinant HCV capsid antigen produced by the method of claim 30.

32. A composition comprising a recombinant HCV capsid antigen of claim 31, wherein said composition is essentially free of procaryotic antigens and other HCV-related proteins.

33. A diagnostic system, in kit form, comprising, in an amount sufficient to perform at least one assay, the composition of an HCV capsid antigen according to claim 32.

34. The diagnostic system according to claim 33, wherein said HCV structural protein is affixed to a solid matrix.

35. A method of assaying a body fluid sample for the presence of antibodies against an HCV capsid antigen which comprises:
a) forming an immunoreaction admixture by admixing said body fluid sample with a composition of claim 32;
b) maintaining said immunoreaction admixture for a time period sufficient for any of said antibodies present to immunoreact with said HCV capsid antigen to form an immunoreaction product; and
c) detecting the presence of any of said immunoreaction product formed and thereby the presence of said antibodies.

36. The method of claim 35, wherein said detecting in step (c) comprises the steps of:
(i) admixing said immunoreaction product formed in step (c) with a labeled specific binding agent to form a labeling admixture, said labeled specific binding agent comprising a specific binding agent and a label;
(ii) maintaining said labeling admixture for a time period sufficient for any of said immunoreaction product present to bind with said labeled specific binding agent to form a labeled product; and
(iii) detecting the presence of any of said labeled product formed, and thereby the presence of said immunoreaction product.

37. The method of claim 36, wherein said specific binding agent is Protein A or at least one of the antibodies anti-human IgG and anti-human IgM.

38. The method of claim 36, wherein said label is a lanthanide chelate, biotin, an enzyme, or a radioactive isotope.

39. A recombinant expression vector comprising a first nucleic acid having the sequence AGGAGGGTTTTTCAT operatively linked to a second nucleic acid consisting of a nucleotide sequence which encodes an HCV nonstructural 794 antigen having the amino acid sequence of SEQ ID NO: 16 or the corresponding sequence from another HCV strain.

40. The vector of claim 39, wherein said expression vector is pGEX7 comprising said first nucleic and second nucleic acids.

41. The vector of claim 40, wherein said vector is pGEX-NS3-794.

42. A procaryotic host cell comprising an expression vector of claim 39.

43. A method of producing an HCV nonstructural 794 antigen which comprises
(a) treating a host cell comprising an expression vector of claim 39 under conditions and for a time effective to express said antigen; and
(b) recovering said antigen.

44. A recombinant HCV nonstructural 794 antigen produced by the method of claim 43.

45. A composition comprising the recombinant HCV nonstructural 794 antigen of claim 44, wherein said composition is essentially free of procaryotic antigens and other HCV-related proteins.

46. A diagnostic system, in kit form, comprising, in an amount sufficient to perform at least one assay, the composition of an HCV nonstructural 794 antigen according to claim 45.

47. The diagnostic system according to claim 46, wherein said HCV nonstructural 794 antigen is affixed to a solid matrix.

48. A method of assaying a body fluid sample for the presence of antibodies against an HCV nonstructural 794 antigen which comprises:
a) forming an immunoreaction admixture by admixing said body fluid sample with a composition of claim 45;
b) maintaining said immunoreaction admixture for a time period sufficient for any of said antibodies present to immunoreact with said HCV nonstructural 794 antigen to form an immunoreaction product; and
c) detecting the presence of any of said immunoreaction product formed and thereby the presence of said antibodies.

49. The method of claim 48, wherein said detecting in step (c) comprises the steps of:
(i) admixing said immunoreaction product formed in step (c) with a labeled specific binding agent to form a labeling admixture, said labeled specific binding agent comprising a specific binding agent and a label;
(ii) maintaining said labeling admixture for a time period sufficient for any of said immunoreaction product present to bind with said labeled specific binding agent to form a labeled product; and
(iii) detecting the presence of any of said labeled product formed, and thereby the presence of said immunoreaction product.

50. The method of claim 49, wherein said specific binding agent is Protein A on at least one of the antibodies anti-human IgG and anti-human IgM.

51. The method of claim 49, wherein said label is a lanthanide chelate, biotin, an enzyme, or a radioactive isotope.

52. A recombinant expression vector comprising a first nucleic acid having the sequence AGGAGGGTTTTTCAT operatively linked to a second nucleic acid consisting of a nucleotide sequence which encodes a CAP-B antigen having the amino acid sequence of SEQ ID NO: 18 or the corresponding sequence from another HCV strain.

53. The vector of claim 52, wherein said expression vector is pGEX7 comprising said first nucleic and second nucleic acids.

54. The vector of claim 53, wherein said vector is pGEX-CAP-B.

55. A procaryotic host cell comprising an expression vector of claim 52.

56. A method of producing an HCV CAP-B antigen which comprises
(a) treating a host cell comprising an expression vector of claim 52 under conditions and for a time effective to express said antigen; and
(b) recovering said antigen.

57. A recombinant HCV CAP-B antigen produced by the method of claim 56.

58. A composition comprising the recombinant HCV CAP-B antigen of claim 57, wherein said composition is essentially free of procaryotic antigens and other HCV-related proteins.

59. A diagnostic system, in kit form, comprising, in an amount sufficient to perform at least one assay, the composition of an HCV CAP-B antigen according to claim 58.

60. The diagnostic system according to claim 59, wherein said HCV CAP-B antigen is affixed to a solid matrix.

61. A method of assaying a body fluid sample for the presence of antibodies against an HCV CAP-B antigen which comprises:
a) forming an immunoreaction admixture by admixing said body fluid sample with a composition of claim 58;
b) maintaining said immunoreaction admixture for a time period sufficient for any of said antibodies present to immunoreact with said HCV CAP-B antigen to form an immunoreaction product; and
c) detecting the presence of any of said immunoreaction product formed and thereby the presence of said antibodies.

62. The method of claim 61, wherein said detecting in step (c) comprises the steps of:
(i) admixing said immunoreaction product formed in step (c) with a labeled specific binding agent to form a labeling admixture, said labeled specific binding agent comprising a specific binding agent and a label;
(ii) maintaining said labeling admixture for a time period sufficient for any of said immunoreaction product present to bind with said labeled specific binding agent to form a labeled product; and
(iii) detecting the presence of any of said labeled product formed, and thereby the presence of said immunoreaction product.

63. The method of claim 62, wherein said specific binding agent is Protein A, or at least one of the antibodies anti-human IgG and anti-human IgM.

64. The method of claim 62, wherein said label is a lanthanide chelate, a biotin, an enzyme, or a radioactive isotope.

65. A composition comprising a recombinant HCV capsid antigen consisting of amino acids 1-120 and a recombinant HCV nonstructural 794 antigen consisting of amino acids of SEQ ID NO: 16 or the corresponding sequence from another HCV strain, wherein said composition is essentially free of procaryotic antigens and other HCV-related proteins.

66. The composition of claim 65 wherein said recombinant HCV capsid antigen consists of amino acids 1-120 of SEQ ID NO:8.

67. The composition of claim 65 wherein said recombinant HCV nonstructural 794 antigen consists of amino acids of SEQ ID NO: 16.

68. The composition of claim 66 wherein said recombinant HCV nonstructural 794 antigen consists of amino acids of SEQ ID NO: 16.

69. The composition of claim 65, wherein the ratio by weight of said capsid antigen to said nonstructural antigen is in a range of about 8:1 to about 1:1.

70. The composition of claim 68, wherein the ratio by weight of said capsid antigen to said nonstructural antigen is in a range of about 8:1 to about 1:1.

71. A diagnostic system, in kit form, comprising, in an amount sufficient to perform at least one assay, the composition of any one of claims 65, 68, 69 or 70.

72. The diagnostic system according to claim 71, wherein said HCV capsid antigen and said HCV nonstructural 794 antigen are affixed to a solid matrix.

73. A method of assaying a body fluid sample for the presence of antibodies against an HCV capsid antigen or an HCV nonstructural antigen, which method comprises:
a) forming an immunoreaction admixture by admixing said body fluid sample with a composition of any one of claims 65, 68, 69 or 70;
b) maintaining said immunoreaction admixture for a time period sufficient for any of said antibodies present to immunoreact with one or more of said antigens to form an immunoreaction product; and
c) detecting the presence of any of said immunoreaction product formed and thereby the presence of said antibodies.

74. The method of claim 73, wherein said detecting in step (c) comprises the steps of:
(i) admixing said immunoreaction product formed in step (c) with a labeled specific binding agent to form a labeling admixture, said labeled specific binding agent comprising a specific binding agent and a label;
(ii) maintaining said labeling admixture for a time period sufficient for any of said immunoreaction product present to bind with said labeled specific binding agent to form a labeled product; and
(iii) detecting the presence of any of said labeled product formed, and thereby the presence of said immunoreaction product.

75. The method of claim 74, wherein said specific binding agent is Protein at least one of the antibodies anti-human IgG and anti-human IgM.

76. The method of claim 74, wherein said label is a lanthanide chelate, biotin, an enzyme, or a radioactive isotope.
